SEQUENCE LISTING (26 sequences)

SEQ ID NO: 1 - DNA, 2919 bp, Homo sapiens
  Exemplary nucleic acid sequence encoding human CXCR5
aaaaaaaaaaagtgatgagttgtgaggcaggtcgcggccctactgcctcaggagacgatgcgcagctcatttgcttaaatttgcagctgacggctgccacctctctagaggcacctggcggggagcctctcaacataagacagtgaccagtctggtgactcacagccggcacagccatgaactacccgctaacgctggaaatggacctcgagaacctggaggacctgttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 2 - DNA, 2896 bp, Homo sapiens
  Exemplary nucleic acid sequence encoding  human CXCR5
ccactctaaggaatgcggtccctttgacaggcgaaaaactgaagttggaaaagacaaagtgatttgttcaaaattgaaatttgaaacttgacatttggtcagtgggccctatgtaggaaaaaacctccaagagagctagggttcctctcagagaggaaagacaggtccttaggtcctcaccctcccgtctccttgcccttgcagttctgggaactggacagattggacaactataacgacacctccctggtggaaaatcatctctgccctgccacagaggggcccctcatggcctccttcaaggccgtgttcgtgcccgtggcctacagcctcatcttcctcctgggcgtgatcggcaacgtcctggtgctggtgatcctggagcggcaccggcagacacgcagttccacggagaccttcctgttccacctggccgtggccgacctcctgctggtcttcatcttgccctttgccgtggccgagggctctgtgggctgggtcctggggaccttcctctgcaaaactgtgattgccctgcacaaagtcaacttctactgcagcagcctgctcctggcctgcatcgccgtggaccgctacctggccattgtccacgccgtccatgcctaccgccaccgccgcctcctctccatccacatcacctgtgggaccatctggctggtgggcttcctccttgccttgccagagattctcttcgccaaagtcagccaaggccatcacaacaactccctgccacgttgcaccttctcccaagagaaccaagcagaaacgcatgcctggttcacctcccgattcctctaccatgtggcgggattcctgctgcccatgctggtgatgggctggtgctacgtgggggtagtgcacaggttgcgccaggcccagcggcgccctcagcggcagaaggcagtcagggtggccatcctggtgacaagcatcttcttcctctgctggtcaccctaccacatcgtcatcttcctggacaccctggcgaggctgaaggccgtggacaatacctgcaagctgaatggctctctccccgtggccatcaccatgtgtgagttcctgggcctggcccactgctgcctcaaccccatgctctacactttcgccggcgtgaagttccgcagtgacctgtcgcggctcctgacgaagctgggctgtaccggccctgcctccctgtgccagctcttccctagctggcgcaggagcagtctctctgagtcagagaatgccacctctctcaccacgttctaggtcccagtgtccccttttattgctgcttttccttggggcaggcagtgatgctggatgctccttccaacaggagctgggatcctaagggctcaccgtggctaagagtgtcctaggagtatcctcatttggggtagctagaggaaccaacccccatttctagaacatccctgccagctcttctgccggccctggggctaggctggagcccagggagcggaaagcagctcaaaggcacagtgaaggctgtccttacccatctgcacccccctgggctgagagaacctcacgcacctcccatcctaatcatccaatgctcaagaaacaacttctacttctgcccttgccaacggagagcgcctgcccctcccagaacacactccatcagcttaggggctgctgacctccacagcttcccctctctcctcctgcccacctgtcaaacaaagccagaagctgagcaccaggggatgagtggaggttaaggctgaggaaaggccagctggcagcagagtgtggccttcggacaactcagtccctaaaaacacagacattctgccaggcccccaagcctgcagtcatcttgaccaagcaggaagctcagactggttgagttcaggtagctgcccctggctctgaccgaaacagcgctgggtccaccccatgtcaccggatcctgggtggtctgcaggcagggctgactctaggtgcccttggaggccagccagtgacctgaggaagcgtgaaggccgagaagcaagaaagaaacccgacagagggaagaaaagagctttcttcccgaaccccaaggagggagatggatcaatcaaacccggcggtcccctccgccaggcgagatggggtggggtggagaactcctagggtggctgggtccaggggatgggaggttgtgggcattgatggggaaggaggctggcttgtcccctcctcactcccttcccataagctatagacccgaggaaactcagagtcggaacggagaaaggtggactggaaggggcccgtgggagtcatctcaaccatcccctccgtggcatcaccttaggcagggaagtgtaagaaacacactgaggcagggaagtccccaggccccaggaagccgtgccctgcccccgtgaggatgtcactcagatggaaccgcaggaagctgctccgtgcttgtttgctcacctggggtgtgggaggcccgtccggcagttctgggtgctccctaccacctccccagcctttgatcaggtggggagtcagggacccctgcccttgtcccactcaagccaagcagccaagctccttgggaggccccactggggaaataacagctgtggctcacgtgagagtgtcttcacggcaggacaacgaggaagccctaagacgtcccttttttctctgagtatctcctcgcaagctgggtaatcgatgggggagtctgaagcagatgcaaagaggcaagaggctggattttgaattttctttttaataaaaaggcacctataaaacaggtcaatacagtacaggcagcacagagacccccggaacaagcctaaaaattgtttcaaaataaaaaccaagaagatgtcttcacatattgtaaaaaaaaaaaaaaaaa

SEQ ID NO: 3 - protein, 327 aa, Homo sapiens
  Exemplary amino acid sequence for human CXCR5 precursor
MASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 4 - protein, 372 aa, Homo sapiens
  Exemplary amino acid sequence for human CXCR5 precursor
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 5 - DNA, 2567 bp, Homo sapiens
  Exemplary nucleic acid sequence encoding human CCR9
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 6 - DNA, 2653 bp, Homo sapiens
  Exemplary nucleic acid sequence encoding human CCR9
gcttcctttctcgtgttgttatcgggtagctgcctgctcagaacccacaaagcctgcccctcatcccaggcagagagcaacccagctctttccccagacactgagagctggtggtgcctgctgtcccagggagagttgcatcgccctccacagagcaggcttgcatctgactgacccaccatgacacccacagacttcacatctcctccaggccccgctccagatcaccttccctcgctggcccaggaatccatctccttccaggaccttagcccaggactaacacaagccctattcctaacatggctgatgactatggctctgaatccacatcttccatggaagactacgttaacttcaacttcactgacttctactgtgagaaaaacaatgtcaggcagtttgcgagccatttcctcccacccttgtactggctcgtgttcatcgtgggtgccttgggcaacagtcttgttatccttgtctactggtactgcacaagagtgaagaccatgaccgacatgttccttttgaatttggcaattgctgacctcctctttcttgtcactcttcccttctgggccattgctgctgctgaccagtggaagttccagaccttcatgtgcaaggtggtcaacagcatgtacaagatgaacttctacagctgtgtgttgctgatcatgtgcatcagcgtggacaggtacattgccattgcccaggccatgagagcacatacttggagggagaaaaggcttttgtacagcaaaatggtttgctttaccatctgggtattggcagctgctctctgcatcccagaaatcttatacagccaaatcaaggaggaatccggcattgctatctgcaccatggtttaccctagcgatgagagcaccaaactgaagtcagctgtcttgaccctgaaggtcattctggggttcttccttcccttcgtggtcatggcttgctgctataccatcatcattcacaccctgatacaagccaagaagtcttccaagcacaaagccctaaaagtgaccatcactgtcctgaccgtctttgtcttgtctcagtttccctacaactgcattttgttggtgcagaccattgacgcctatgccatgttcatctccaactgtgccgtttccaccaacattgacatctgcttccaggtcacccagaccatcgccttcttccacagttgcctgaaccctgttctctatgtttttgtgggtgagagattccgccgggatctcgtgaaaaccctgaagaacttgggttgcatcagccaggcccagtgggtttcatttacaaggagagagggaagcttgaagctgtcgtctatgttgctggagacaacctcaggagcactctccctctgaggggtcttctctgaggtgcatggttcttttggaagaaatgagaaatacagaaacagtttccccactgatgggaccagagagagtgaaagagaaaagaaaactcagaaagggatgaatctgaactatatgattacttgtagtcagaatttgccaaagcaaatatttcaaaatcaactgactagtgcaggaggctgttgattggctcttgactgtgatgcccgcaattctcaaaggaggactaaggaccggcactgtggagcaccctggctttgccactcgccggagcatcaatgccgctgcctctggaggagcccttggattttctccatgcactgtgaacttctgtggcttcagttctcatgctgcctcttccaaaaggggacacagaagcactggctgctgctacagaccgcaaaagcagaaagtttcgtgaaaatgtccatctttgggaaattttctaccctgctcttgagcctgataacccatgccaggtcttatagattcctgatctagaacctttccaggcaatctcagacctaatttccttctgttctccttgttctgttctgggccagtgaaggtccttgttctgattttgaaacgatctgcaggtcttgccagtgaacccctggacaactgaccacacccacaaggcatccaaagtctgttggcttccaatccatttctgtgtcctgctggaggttttaacctagacaaggattccgcttattccttggtatggtgacagtgtctctccatggcctgagcagggagattataacagctgggttcgcaggagccagccttggccctgttgtaggcttgttctgttgagtggcacttgctttgggtccaccgtctgtctgctccctagaaaatgggctggttcttttggccctcttctttctgaggcccactttattctgaggaatacagtgagcagatatgggcagcagccaggtagggcaaaggggtgaagcgcaggccttgctggaaggctatttacttccatgcttctccttttcttactctatagtggcaacattttaaaagcttttaacttagagattaggctgaaaaaaataagtaatggaattcacctttgcatcttttgtgtctttcttatcatgatttggcaaaatgcatcacctttgaaaatatttcacatattggaaaagtgctttttaatgtgtatatgaagcattaattacttgtcactttctttaccctgtctcaatattttaagtgtgtgcaattaaagatcaaatagatacatt

SEQ ID NO: 7 - protein, 369 aa, Homo sapiens
  Exemplary amino acid sequence for human CCR9 precursor
MTPTDFTSPIPNMADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 8 - protein, 357 aa, Homo sapiens
  Exemplary amino acid sequence for human CCR9 precursor
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 9 - DNA, 6082 bp, Homo sapiens
  Exemplary nucleic acid sequence encoding human alpha-4
ataacgtctttgtcactaaaatgttccccaggggccttcggcgagtctttttgtttggttttttgtttttaatctgtggctcttgataatttatctagtggttgcctacacctgaaaaacaagacacagtgtttaactatcaacgaaagaactggacggctccccgccgcagtcccactccccgagtttgtggctggcatttgggccacgccgggctgggcggtcacagcgaggggcgcgcagtttggggtcacacagctccgcttctaggccccaaccaccgttaaaaggggaagcccgtgccccatcaggtccgctcttgctgagcccagagccatcccgcgctctgcgggctgggaggcccgggccaggacgcgagtcctgcgcagccgaggttccccagcgccccctgcagccgcgcgtaggcagagacggagcccggccctgcgcctccgcaccacgcccgggaccccacccagcggcccgtacccggagaagcagcgcgagcacccgaagctcccggctggcggcagaaaccgggagtggggccgggcgagtgcgcggcatcccaggccggcccgaacgctccgcccgcggtgggccgacttcccctcctcttccctctctccttcctttagcccgctggcgccggacacgctgcgcctcatctcttggggcgttcttccccgttggccaaccgtcgcatcccgtgcaactttggggtagtggccgtttagtgttgaatgttccccaccgagagcgcatggcttgggaagcgaggcgcgaacccggcccccgaagggccgccgtccgggagacggtgatgctgttgctgtgcctgggggtcccgaccggccgcccctacaacgtggacactgagagcgcgctgctttaccagggcccccacaacacgctgttcggctactcggtcgtgctgcacagccacggggcgaaccgatggctcctagtgggtgcgcccactgccaactggctcgccaacgcttcagtgatcaatcccggggcgatttacagatgcaggatcggaaagaatcccggccagacgtgcgaacagctccagctgggtagccctaatggagaaccttgtggaaagacttgtttggaagagagagacaatcagtggttgggggtcacactttccagacagccaggagaaaatggatccatcgtgacttgtgggcatagatggaaaaatatattttacataaagaatgaaaataagctccccactggtggttgctatggagtgccccctgatttacgaacagaactgagtaaaagaatagctccgtgttatcaagattatgtgaaaaaatttggagaaaattttgcatcatgtcaagctggaatatccagtttttacacaaaggatttaattgtgatgggggccccaggatcatcttactggactggctctctttttgtctacaatataactacaaataaatacaaggcttttttagacaaacaaaatcaagtaaaatttggaagttatttaggatattcagtcggagctggtcattttcggagccagcatactaccgaagtagtcggaggagctcctcaacatgagcagattggtaaggcatatatattcagcattgatgaaaaagaactaaatatcttacatgaaatgaaaggtaaaaagcttggatcgtactttggagcttctgtctgtgctgtggacctcaatgcagatggcttctcagatctgctcgtgggagcacccatgcagagcaccatcagagaggaaggaagagtgtttgtgtacatcaactctggctcgggagcagtaatgaatgcaatggaaacaaacctcgttggaagtgacaaatatgctgcaagatttggggaatctatagttaatcttggcgacattgacaatgatggctttgaagatgttgctatcggagctccacaagaagatgacttgcaaggtgctatttatatttacaatggccgtgcagatgggatctcgtcaaccttctcacagagaattgaaggacttcagatcagcaaatcgttaagtatgtttggacagtctatatcaggacaaattgatgcagataataatggctatgtagatgtagcagttggtgcttttcggtctgattctgctgtcttgctaaggacaagacctgtagtaattgttgacgcttctttaagccaccctgagtcagtaaatagaacgaaatttgactgtgttgaaaatggatggccttctgtgtgcatagatctaacactttgtttctcatataagggcaaggaagttccaggttacattgttttgttttataacatgagtttggatgtgaacagaaaggcagagtctccaccaagattctatttctcttctaatggaacttctgacgtgattacaggaagcatacaggtgtccagcagagaagctaactgtagaacacatcaagcatttatgcggaaagatgtgcgggacatcctcaccccaattcagattgaagctgcttaccaccttggtcctcatgtcatcagtaaacgaagtacagaggaattcccaccacttcagccaattcttcagcagaagaaagaaaaagacataatgaaaaaaacaataaactttgcaaggttttgtgcccatgaaaattgttctgctgatttacaggtttctgcaaagattgggtttttgaagccccatgaaaataaaacatatcttgctgttgggagtatgaagacattgatgttgaatgtgtccttgtttaatgctggagatgatgcatatgaaacgactctacatgtcaaactacccgtgggtctttatttcattaagattttagagctggaagagaagcaaataaactgtgaagtcacagataactctggcgtggtacaacttgactgcagtattggctatatatatgtagatcatctctcaaggatagatattagctttctcctggatgtgagctcactcagcagagcggaagaggacctcagtatcacagtgcatgctacctgtgaaaatgaagaggaaatggacaatctaaagcacagcagagtgactgtagcaatacctttaaaatatgaggttaagctgactgttcatgggtttgtaaacccaacttcatttgtgtatggatcaaatgatgaaaatgagcctgaaacgtgcatggtggagaaaatgaacttaactttccatgttatcaacactggcaatagtatggctcccaatgttagtgtggaaataatggtaccaaattcttttagcccccaaactgataagctgttcaacattttggatgtccagactactactggagaatgccactttgaaaattatcaaagagtgtgtgcattagagcagcaaaagagtgcaatgcagaccttgaaaggcatagtccggttcttgtccaagactgataagaggctattgtactgcataaaagctgatccacattgtttaaatttcttgtgtaattttgggaaaatggaaagtggaaaagaagccagtgttcatatccaactggaaggccggccatccattttagaaatggatgagacttcagcactcaagtttgaaataagagcaacaggttttccagagccaaatccaagagtaattgaactaaacaaggatgagaatgttgcgcatgttctactggaaggactacatcatcaaagacccaaacgttatttcaccatagtgattatttcaagtagcttgctacttggacttattgtacttctgttgatctcatatgttatgtggaaggctggcttctttaaaagacaatacaaatctatcctacaagaagaaaacagaagagacagttggagttatatcaacagtaaaagcaatgatgattaaggacttctttcaaattgagagaatggaaaacagactcaggttgtagtaaagaaatttaaaagacactgtttacaagaaaaaatgaattttgtttggacttcttttactcatgatcttgtgacatattatgtcttcatgcaaggggaaaatctcagcaatgattactctttgagatagaagaactgcaaaggtaataatacagccaaagataatctctcagcttttaaatgggtagagaaacactaaagcattcaatttattcaagaaaagtaagcccttgaagatatcttgaaatgaaagtataactgagttaaattatactggagaagtcttagacttgaaatactacttaccatatgtgcttgcctcagtaaaatgaaccccactgggtgggcagaggttcatttcaaatacatctttgatacttgttcaaaatatgttctttaaaaatataattttttagagagctgttcccaaattttctaacgagtggaccattatcactttaaagccctttatttataatacatttcctacgggctgtgttccaacaaccattttttttcagcagactatgaatattatagtattataggccaaactggcaaacttcagactgaacatgtacactggtttgagcttagtgaaattacttctggataattatttttttataattatggatttcaccatctttctttctgtatatatacatgtgtttttatgtaggtatatatttaccattcttcctatctattcttcctataacacacctttatcaagcatacccaggagtaatcttcaaatcttttgttatattctgaaacaaaagattgtgagtgttgcactttacctgatacacgctgatttagaaaatacagaaaccatacctcactaataactttaaaatcaaagctgtgcaaagactagggggcctatacttcatatgtattatgtactatgtaaaatattgactatcacacaactatttccttggatgtaattctttgttaccctttacaagtataagtgttaccttacatggaaacgaagaaacaaaattcataaatttaaattcataaatttagctgaaagatactgattcaatttgtatacagtgaatataaatgagacgacagcaaaattttcatgaaatgtaaaatatttttatagtttgttcatactatatgaggttctattttaaatgactttctggattttaaaaaatttctttaaatacaatcatttttgtaatatttattttatgcttatgatctagataattgcagaatatcattttatctgactctgccttcataagagagctgtggccgaattttgaacatctgttatagggagtgatcaaattagaaggcaatgtggaaaaacaattctgggaaagatttctttatatgaagtccctgccactagccagccatcctaattgatgaaagttatctgttcacaggcctgcagtgatggtgaggaatgttctgagatttgcgaaggcatttgagtagtgaaatgtaagcacaaaacctcctgaacccagagtgtgtatacacaggaataaactttatgacatttatgtatttttaaaaaactttgtatcgttataaaaaggctagtcattctttcaggagaacatctaggatcatagatgaaaaatcaagccccgatttagaactgtcttctccaggatggtctctaaggaaatttacatttggttctttcctactcagaactactcagaaacaactatatatttcaggttatctgagcacagtgaaagcagagtactatggttgtccaacacaggcctctcagatacaaggggaacacaattacatattgggctagattttgcccagttcaaaatagtatttgttatcaacttactttgttacttgtatcatgaattttaaaaccctaccactttaagaagacagggatgggttattcttttttggcaggtaggctatataactatgtgattttgaaatttaactgctctggattagggagcagtgaatcaaggcagacttatgaaatctgtattatatttgtaacagaatataggaaatttaacataattgatgagctcaaatcctgaaaaatgaaagaatccaaattatttcagaattatctaggttaaatattgatgtattatgatggttgcaaagtttttttgtgtgtccaataaacacattgtaaaaaaaa

SEQ ID NO: 10 - DNA, 2878 bp, Homo sapiens
  Exemplary nucleic acid sequence encoding human beta-7
aaatcttccccaccctggggagtgtcacttcctcctctgccgtctcccagatcagtacacaaaggctgctgctgccgccagaggaaggactgctctgcacgcacctatgtggaaactaaagcccagagagaaagtctgacttgccccacagccagtgagtgactgcagcagcaccagaatctggtctgtttcctgtttggctcttctaccactacggcttgggatctcgggcatggtggctttgccaatggtccttgttttgctgctggtcctgagcagaggtgagagtgaattggacgccaagatcccatccacaggggatgccacagaatggcggaatcctcacctgtccatgctggggtcctgccagccagccccctcctgccagaagtgcatcctctcacaccccagctgtgcatggtgcaagcaactgaacttcaccgcgtcgggagaggcggaggcgcggcgctgcgcccgacgagaggagctgctggctcgaggctgcccgctggaggagctggaggagccccgcggccagcaggaggtgctgcaggaccagccgctcagccagggcgcccgcggagagggtgccacccagctggcgccgcagcgggtccgggtcacgctgcggcctggggagccccagcagctccaggtccgcttccttcgtgctgagggatacccggtggacctgtactaccttatggacctgagctactccatgaaggacgacctggaacgcgtgcgccagctcgggcacgctctgctggtccggctgcaggaagtcacccattctgtgcgcattggttttggttcctttgtggacaaaacggtgctgccctttgtgagcacagtaccctccaaactgcgccacccctgccccacccggctggagcgctgccagtcaccattcagctttcaccatgtgctgtccctgacgggggacgcacaagccttcgagcgggaggtggggcgccagagtgtgtccggcaatctggactcgcctgaaggtggcttcgatgccattctgcaggctgcactctgccaggagcagattggctggagaaatgtgtcccggctgctggtgttcacttcagacgacacattccatacagctggggacgggaagttgggcggcattttcatgcccagtgatgggcactgccacttggacagcaatggcctctacagtcgcagcacagagtttgactacccttctgtgggtcaggtagcccaggccctctctgcagcaaatatccagcccatctttgctgtcaccagtgccgcactgcctgtctaccaggagctgagtaaactgattcctaagtctgcagttggggagctgagtgaggactccagcaacgtggtacagctcatcatggatgcttataatagcctgtcttccaccgtgacccttgaacactcttcactccctcctggggtccacatttcttacgaatcccagtgtgagggtcctgagaagagggagggtaaggctgaggatcgaggacagtgcaaccacgtccgaatcaaccagacggtgactttctgggtttctctccaagccacccactgcctcccagagccccatctcctgaggctccgggcccttggcttctcagaggagctgattgtggagttgcacacgctgtgtgactgtaattgcagtgacacccagccccaggctccccactgcagtgatggccagggacacctacaatgtggtgtatgcagctgtgcccctggccgcctaggtcggctctgtgagtgctctgtggcagagctgtcctccccagacctggaatctgggtgccgggctcccaatggcacagggcccctgtgcagtggaaagggtcactgtcaatgtggacgctgcagctgcagtggacagagctctgggcatctgtgcgagtgtgacgatgccagctgtgagcgacatgagggcatcctctgcggaggctttggtcgctgccaatgtggagtatgtcactgtcatgccaaccgcacgggcagagcatgcgaatgcagtggggacatggacagttgcatcagtcccgagggagggctctgcagtgggcatggacgctgcaaatgcaaccgctgccagtgcttggacggctactatggtgctctatgcgaccaatgcccaggctgcaagacaccatgcgagagacaccgggactgtgcagagtgtggggccttcaggactggcccactggccaccaactgcagtacagcttgtgcccataccaatgtgaccctggccttggcccctatcttggatgatggctggtgcaaagagcggaccctggacaaccagctgttcttcttcttggtggaggatgacgccagaggcacggtcgtgctcagagtgagaccccaagaaaagggagcagaccacacgcaggccattgtgctgggctgcgtagggggcatcgtggcagtggggctggggctggtcctggcttaccggctctcggtggaaatctatgaccgccgggaatacagtcgctttgagaaggagcagcaacaactcaactggaagcaggacagtaatcctctctacaaaagtgccatcacgaccaccatcaatcctcgctttcaagaggcagacagtcccactctctgaaggagggagggacacttacccaaggctcttctccttggaggacagtgggaactggagggtgagaggaagggtgggtctgtaagaccttggtaggggactaattcactggcgaggtgcggccaccaccctacttcattttcagagtgacacccaagagggctgcttcccatgcctgcaaccttgcatccatctgggctaccccacccaagtatacaataaagtcttacctcagaccacaaaaaaaaaaaa

SEQ ID NO: 11 - protein, 1032 aa, Homo sapiens [sequence omitted]

SEQ ID NO: 12 - protein, 798 aa, Homo sapiens
  Exemplary amino acid sequence for human beta-7 precursor
MVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADHTQAIVLGCVGGIVAVGLGLVLAYRLSVEIYDRREYSRFEKEQQQLNWKQDSNPLYKSAITTTINPRFQEADSPTL

SEQ ID NO: 13 - DNA, 1244 bp, Homo sapiens
  Exemplary nucleic acid sequence encoding human CCR10
agagatggggacggaggccacagagcaggtttcctggggccattactctggggatgaagaggacgcatactcggctgagccactgccggagctttgctacaaggccgatgtccaggccttcagccgggccttccaacccagtgtctccctgaccgtggctgcgctgggtctggccggcaatggcctggtcctggccacccacctggcagcccgacgcgcagcgcgctcgcccacctctgcccacctgctccagctggccctggccgacctcttgctggccctgactctgcccttcgcggcagcaggggctcttcagggctggagtctgggaagtgccacctgccgcaccatctctggcctctactcggcctccttccacgccggcttcctcttcctggcctgtatcagcgccgaccgctacgtggccatcgcgcgagcgctcccagccgggccgcggccctccactcccggccgcgcacacttggtctccgtcatcgtgtggctgctgtcactgctcctggcgctgcctgcgctgctcttcagccaggatgggcagcgggaaggccaacgacgctgtcgcctcatcttccccgagggcctcacgcagacggtgaagggggcgagcgccgtggcgcaggtggccctgggcttcgcgctgccgctgggcgtcatggtagcctgctacgcgcttctgggccgcacgctgctggccgccagggggcccgagcgccggcgtgcgctgcgcgtcgtggtggctctggtggcggccttcgtggtgctgcagctgccctacagcctcgccctgctgctggatactgccgatctactggctgcgcgcgagcggagctgccctgccagcaaacgcaaggatgtcgcactgctggtgaccagcggcttggccctcgcccgctgtggcctcaatcccgttctctacgccttcctgggcctgcgcttccgccaggacctgcggaggctgctacggggtgggagctgcccctcagggcctcaaccccgccgcggctgcccccgccggccccgcctttcttcctgctcagctcccacggagacccacagtctctcctgggacaactagggctgcgaatctagaggagggggcaggctgagggtcgtgggaaaggggagtaggtgggggaacactgagaaagaggcagggacctaaagggactacctctgtgccttgccacattaaattgataacatggaaatgagatgcaacccaacaa

SEQ ID NO: 14 - DNA, 1244 bp, Homo sapiens
  Exemplary nucleic acid sequence encoding human CCR10
agagatggggacggaggccacagagcaggtttcctggggccattactctggggatgaagaggacgcatactcggctgagccactgccggagctttgctacaaggccgatgtccaggccttcagccgggccttccaacccagtgtctccctgaccgtggctgcgctgggtctggccggcaatggcctggtcctggccacccacctggcagcccgacgcgcagcgcgctcgcccacctctgcccacctgctccagctggccctggccgacctcttgctggccctgactctgcccttcgcggcagcaggggctcttcagggctggagtctgggaagtgccacctgccgcaccatctctggcctctactcggcctccttccacgccggcttcctcttcctggcctgtatcagcgccgaccgctacgtggccatcgcgcgagcgctcccagccgggccgcggccctccactcccggccgcgcacacttggtctccgtcatcgtgtggctgctgtcactgctcctggcgctgcctgcgctgctcttcagccaggatgggcagcgggaaggccaacgacgctgtcgcctcatcttccccgagggcctcacgcagacggtgaagggggcgagcgccgtggcgcaggtggccctgggcttcgcgctgccgctgggcgtcatggtagcctgctacgcgcttctgggccgcacgctgctggccgccagggggcccgagcgccggcgtgcgctgcgcgtcgtggtggctctggtggcggccttcgtggtgctgcagctgccctacagcctcgccctgctgctggatactgccgatctactggctgcgcgcgagcggagctgccctgccagcaaacgcaaggatgtcgcactgctggtgaccagcggcttggccctcgcccgctgtggcctcaatcccgttctctacgccttcctgggcctgcgcttccgccaggacctgcggaggctgctacggggtgggagctcgccctcagggcctcaaccccgccgcggctgcccccgccggccccgcctttcttcctgctcagctcccacggagacccacagtctctcctgggacaactagggctgcgaatctagaggagggggcaggctgagggtcgtgggaaaggggagtaggtgggggaacactgagaaagaggcagggacctaaagggactacctctgtgccttgccacattaaattgataacatggaaatgaaaaaaaaaaaaaaaa

SEQ ID NO: 15 - protein, 362 aa, Homo sapiens
  Exemplary amino acid sequence for human CCR10 precursor
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSCPSGPQPRRGCPRRPRLSSCSAPTETHSLSWDN

SEQ ID NO: 16 - protein, 362 aa, Homo sapiens; identical to SEQ ID NO: 15
  Exemplary amino acid sequence for human CCR10 precursor

SEQ ID NO: 17 - DNA, 1487 bp, Homo sapiens
  Exemplary nucleic acid sequence encoding human CCR8
tttgtagtgggaggatacctccagagaggctgctgctcattgagctgcactcacatgaggatacagactttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccatatatgttgttgccaacacttggaacacaatgactaaagacatagttgtgcatgcctggcacaacatcaagcctgtgattgtgtttattgatgatgttgaacaagtggtaactttaaaggattctgtatgccaagtgaaaaaaaaagatgtctgacctccttacatat

SEQ ID NO: 18 - DNA, 1420 bp, Homo sapiens
  Exemplary nucleic acid sequence encoding human CCR8
ctttgtgaagaaggaattggcaacactgaaacctccagaacaaaggctgtcactaaggtcccgctgccttgatggattatacacttgacctcagtgtgacaacagtgaccgactactactaccctgatatcttctcaagcccctgtgatgcggaacttattcagacaaatggcaagttgctccttgctgtcttttattgcctcctgtttgtattcagtcttctgggaaacagcctggtcatcctggtccttgtggtctgcaagaagctgaggagcatcacagatgtatacctcttgaacctggccctgtctgacctgctttttgtcttctccttcccctttcagacctactatctgctggaccagtgggtgtttgggactgtaatgtgcaaagtggtgtctggcttttattacattggcttctacagcagcatgtttttcatcaccctcatgagtgtggacaggtacctggctgttgtccatgccgtgtatgccctaaaggtgaggacgatcaggatgggcacaacgctgtgcctggcagtatggctaaccgccattatggctaccatcccattgctagtgttttaccaagtggcctctgaagatggtgttctacagtgttattcattttacaatcaacagactttgaagtggaagatcttcaccaacttcaaaatgaacattttaggcttgttgatcccattcaccatctttatgttctgctacattaaaatcctgcaccagctgaagaggtgtcaaaaccacaacaagaccaaggccatcaggttggtgctcattgtggtcattgcatctttacttttctgggtcccattcaacgtggttcttttcctcacttccttgcacagtatgcacatcttggatggatgtagcataagccaacagctgacttatgccacccatgtcacagaaatcatttcctttactcactgctgtgtgaaccctgttatctatgcttttgttggggagaagttcaagaaacacctctcagaaatatttcagaaaagttgcagccaaatcttcaactacctaggaagacaaatgcctagggagagctgtgaaaagtcatcatcctgccagcagcactcctcccgttcctccagcgtagactacattttgtgaggatcaatgaagactaaatataaaaaacattttcttgaatggcatgctagtagcagtgagcaaaggtgtgggtgtgaaaggtttccaaaaaaagttcagcatgaaggatgccgtgtgtgttgttgccaacacttggaacacgatgactggggacgtggttgtgcatgcctggcacaacatcaagcctgtgattgtgtttattgatgatgttgaacaagtggtggctttggaggattctgtatgccaagtgaaaggggagatgtctgacctccttcatatag

SEQ ID NO: 19 - protein, 355 aa, Homo sapiens
  Exemplary amino acid sequence for human CCR8 precursor
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 20 - protein, 355 aa, Homo sapiens; identical to SEQ ID NO: 19
  Exemplary amino acid sequence for human CCR8 precursor

SEQ ID NO: 21 - DNA, 1657 bp, Homo sapiens
  Exemplary nucleic acid sequence encoding human CCR4
gctcacaggaagccacgcacccttgaaaggcaccgggtccttcttagcatcgtgcttcctgagcaagcctggcattgcctcacagaccttcctcagagccgctttcagaaaagcaagctgcttctggttgggcccagacctgccttgaggagcctgtagagttaaaaaatgaaccccacggatatagcagacaccaccctcgatgaaagcatatacagcaattactatctgtatgaaagtatccccaagccttgcaccaaagaaggcatcaaggcatttggggagctcttcctgcccccactgtattccttggtttttgtatttggtctgcttggaaattctgtggtggttctggtcctgttcaaatacaagcggctcaggtccatgactgatgtgtacctgctcaaccttgccatctcggatctgctcttcgtgttttccctccctttttggggctactatgcagcagaccagtgggtttttgggctaggtctgtgcaagatgatttcctggatgtacttggtgggcttttacagtggcatattctttgtcatgctcatgagcattgatagatacctggcaattgtgcacgcggtgttttccttgagggcaaggaccttgacttatggggtcatcaccagtttggctacatggtcagtggctgtgttcgcctcccttcctggctttctgttcagcacttgttatactgagcgcaaccatacctactgcaaaaccaagtactctctcaactccacgacgtggaaggttctcagctccctggaaatcaacattctcggattggtgatccccttagggatcatgctgttttgctactccatgatcatcaggaccttgcagcattgtaaaaatgagaagaagaacaaggcggtgaagatgatctttgccgtggtggtcctcttccttgggttctggacaccttacaacatagtgctcttcctagagaccctggtggagctagaagtccttcaggactgcacctttgaaagatacttggactatgccatccaggccacagaaactctggcttttgttcactgctgccttaatcccatcatctacttttttctgggggagaaatttcgcaagtacatcctacagctcttcaaaacctgcaggggcctttttgtgctctgccaatactgtgggctcctccaaatttactctgctgacacccccagctcatcttacacgcagtccaccatggatcatgatctccatgatgctctgtagaaaaatgaaatggtgaaatgcagagtcaatgaactttccacattcagagcttacttaaaattgtattttagtaagagattcctgagccagtgtcaggaggaaggcttacacccacagtggaaagacagcttctcatcctgcaggcagctttttctctcccactagacaagtccagcctggcaagggttcacctgggctgaggcatccttcctcacaccaggcttgcctgcaggcatgagtcagtctgatgagaactctgagcagtgcttgaatgaagttgtaggtaatattgcaaggcaaagactattcccttctaacctgaactgatgggtttctccagagggaattgcagagtactggctgatggagtaaatcgctaccttttgctgtggcaaatgggccctct

SEQ ID NO: 22 - protein, 360 aa, Homo sapiens
  Exemplary amino acid sequence for human CCR4 precursor
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 23 - DNA, 2638 bp, Homo sapiens
  Exemplary nucleic acid sequence encoding human CLA
aatcatccgagaaccttggagggtggacagtgccccttttacagatgagaaaactgaggcttgaaggggagaagcagctgcctctggcggcatggcttctggctgcaggatgcccatggagttcgtggtgaccctaggcctgtgtctcggcttcctttgctgaacttgaacaggaagatggcagtgggggccagtggtctagaaggagataagatggctggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgatagcggaatccttcaaggtttcaaggctgtctccttcaggcagccttcccggaattctccatccctcagtgcaggatgggggctggtcctcagctgtctgccctcagcccctggccccccaggaagcctctttcatgggctgttaggttgacttcagttttgcctcttggacaacagggggtcttgtacatccttgggtgaccaggaaaagttcaggctatggggggccaaagggagggctgccccttccccaccagtgaccactttattccacttcctccattacccagttttggcccacagagtttggtcccccccaaacctcggaccaatatccctctaaacatcaatctatcctcctgttaaagaaaaaaaaaaa

SEQ ID NO: 24 - DNA, 2573 bp, Homo sapiens
  Exemplary nucleic acid sequence encoding human CLA
acacacagccattgggggttgctcggatccgggactgccgcagggggtgccacagcagtgcctggcagcgtgggctgggaccttgtcactaaagcagagaagccacttcttctgggcccacgaggcagctgtcccatgctctgctgagcacggtggtgccatgcctctgcaactcctcctgttgctgatcctactgggccctggcaacagcttgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactcaaccagtgcccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcagtgcctgctggccatcctaatcttggcgctggtggccactatcttcttcgtgtgcactgtggtgctggcggtccgcctctcccgcaagggccacatgtaccccgtgcgtaattactcccccaccgagatggtctgcatctcatccctgttgcctgatgggggtgaggggccctctgccacagccaatgggggcctgtccaaggccaagagcccgggcctgacgccagagcccagggaggaccgtgagggggatgacctcaccctgcacagcttcctcccttagctcactctgccatctgttttggcaagaccccacctccacgggctctcctgggccacccctgagtgcccagaccccattccacagctctgggcttcctcggagacccctggggatggggatcttcagggaaggaactctggccacccaaacaggacaagagcagcctggggccaagcagacgggcaagtggagccacctctttcctccctccgcggatgaagcccagccacatttcagccgaggtccaaggcaggaggccatttacttgagacagattctctcctttttcctgtcccccatcttctctgggtccctctaacatctcccatggctctccccgcttctcctggtcactggagtctcctccccatgtacccaaggaagatggagctcccccatcccacacgcactgcactgccattgtcttttggttgccatggtcaccaaacaggaagtggacattctaagggaggagtactgaagagtgacggacttctgaggctgtttcctgctgctcctctgacttggggcagcttgggtcttcttgggcacctctctgggaaaacccagggtgaggttcagcctgtgagggctgggatgggtttcgtgggcccaagggcagacctttctttgggactgtgtggaccaaggagcttccatctagtgacaagtgacccccagctatcgcctcttgccttcccctgtggccactttccagggtggactctgtcttgttcactgcagtatcccaactgcaggtccagtgcaggcaataaatatgtgatggacaaacgatagcggaatccttcaaggtttcaaggctgtctccttcaggcagccttcccggaattctccatccctcagtgcaggatgggggctggtcctcagctgtctgccctcagcccctggccccccaggaagcctctttcatgggctgttaggttgacttcagttttgcctcttggacaacagggggtcttgtacatccttgggtgaccaggaaaagttcaggctatggggggccaaagggagggctgccccttccccaccagtgaccactttattccacttcctccattacccagttttggcccacagagtttggtcccccccaaacctcggaccaatatccctctaaacatcaatctatcctcctgttaaagaaaaaaaaaaa

SEQ ID NO: 25 - protein, 428 aa, Homo sapiens
  Exemplary amino acid sequence for human CLA precursor
MAVGASGLEGDKMAGAMPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

SEQ ID NO: 26 - protein, 412 aa, Homo sapiens
  Exemplary amino acid sequence for human CLA precursor
MPLQLLLLLILLGPGNSLQLWDTWADEAEKALGPLLARDRRQATEYEYLDYDFLPETEPPEMLRNSTDTTPLTGPGTPESTTVEPAARRSTGLDAGGAVTELTTELANMGNLSTDSAAMEIQTTQPAATEAQTTQPVPTEAQTTPLAATEAQTTRLTATEAQTTPLAATEAQTTPPAATEAQTTQPTGLEAQTTAPAAMEAQTTAPAAMEAQTTPPAAMEAQTTQTTAMEAQTTAPEATEAQTTQPTATEAQTTPLAAMEALSTEPSATEALSMEPTTKRGLFIPFSVSSVTHKGIPMAASNLSVNYPVGAPDHISVKQCLLAILILALVATIFFVCTVVLAVRLSRKGHMYPVRNYSPTEMVCISSLLPDGGEGPSATANGGLSKAKSPGLTPEPREDREGDDLTLHSFLP

(19) United States
(12) Patent Application Publication
(10) Pub. No.: US 2021/0254003 A1
(43) Pub. Date: Aug. 19, 2021

(54) CAR-T LYMPHOCYTES ENGINEERED TO HOME TO LYMPH NODE B CELL ZONE, SKIN, OR GASTROINTESTINAL TRACT

(71) Applicant: CELGENE CORPORATION, Summit, NJ (US)

(72) Inventors: Bitao LIANG, Closter, NJ (US); Wei LIU, Bridgewater, NJ (US); Tianjian LI, Belle Mead, NJ (US)

(73) Assignee: CELGENE CORPORATION, Summit, NJ (US)

(21) Appl. No.: 17/039,507

(22) Filed: Sep. 30, 2020

Related U.S. Application Data
(62) Division of application No. 15/502,752, filed on Feb. 8, 2017, now abandoned.
(60) Provisional application No. 62/036,447, filed on Aug. 12, 2014.

(51) Int. Cl. C12N 5/0783; A61K 39/00; C07K 14/725; C07K 14/705; C07K 14/715; C07K 16/30

(52) U.S. Cl. CPC C12N 5/0638; C12N 2501/39; A61K 39/001129; A61K 39/001182; C07K 14/7051; C07K 14/70546; C07K 14/7158; C07K 16/3007; A61K 2039/5156; C12N 2501/515; C12N 2501/21; C12N 2510/00; A61K 2039/5158; C12N 2501/385; A61K 39/0011

(57) ABSTRACT

In one embodiment, provided herein are cells, e.g., T cells expressing receptors that that cause a cell expressing the receptors to home to specific anatomical regions, e.g., the B cell zone of lymph nodes, the gastrointestinal tract, or the skin. Also provided herein is use of such cells, e.g., T lymphocytes, to treat diseases such as cancer.

This application claims benefit of U.S. Provisional Patent Application No. 62/036,447, filed Aug. 12, 2014, the disclosure of which is incorporated by reference herein in its entirety.

### 1. FIELD

The disclosure herein relates to the field of immunology, and more specifically, to the modification of T lymphocytes or other immune cells.

### 2. BACKGROUND

T lymphocytes recognize and interact with specific antigens, including tumor-associated or tumor-specific antigens. Because T lymphocytes are able to kill tumor cells, the last 25 years has seen a great deal of interest in targeting tumor cells with T lymphocytes, either antigen-specific T lymphocytes, or T lymphocytes genetically modified to express one or more chimeric antigen receptors (CARs; see, e.g., Eshhar, U.S. Pat. No. 7,741,465; Eshhar, U.S. Patent Application Publication No. 2012/0093842). However, given the difficulty of targeting CAR T cells to the necessary areas of the body, it is desirable to find new methods to enhance the homing of CAR T cells.

### 3. SUMMARY

Provided herein are genetically modified cells, for example immune cells, such as T lymphocytes, e.g., human T lymphocytes, that comprise a receptor that causes a cell expressing said receptor to home to a particular anatomical zone, a particular tissue, or a particular type of cell, e.g., the B cell zone of the lymph nodes, gastrointestinal tract, or skin. In a specific embodiment, the genetically modified cells provided herein are T lymphocytes, for example, primary T lymphocytes. In certain embodiments, the genetically modified cells express a chimeric antigen receptor (CAR). Without wishing to be bound by any particular mechanism or theory, it is thought that when the genetically modified cells herein express receptors that cause a cell expressing said receptors to home to a particular zone, they are more likely to be exposed to native antigen, where the cells, for example, cells expressing a CAR, are capable of being activated.

In a first aspect, provided herein is a cell, e.g., a T lymphocyte, for example, a primary T lymphocyte, or a population of such cells, that comprises, e.g., has been engineered to express, a homing receptor and a CAR. In certain embodiments, the homing receptor is not normally or endogenously expressed in the T lymphocyte. In certain embodiments, the homing receptor is a B cell zone homing receptor. In a specific embodiment, the B cell zone homing receptor is CXCR5. In a more specific embodiment, the cell, or population of cells comprising the homing receptor, e.g., the B cell zone homing receptor, additionally lacks expression of a T cell zone homing receptor, e.g., CXCR7, or has reduced expression of said T cell zone homing receptor as compared to normal T cells, e.g., the cell, or population of cells, is engineered to express a T cell homing receptor at a lower level than normal T cells.

In certain embodiments, the homing receptor is a gastrointestinal homing receptor. In a specific embodiment, the gastrointestinal homing receptor is integrin α4β7 (also known as lymphocyte Peyer patch adhesion molecule). In other specific embodiments, the gastrointestinal homing receptor is CCR9 (C-C chemokines receptor type 9). In certain embodiments, the cell, e.g., a T lymphocyte, for example, a primary T lymphocyte, or population of such cells, comprises, e.g., has been engineered to express more than one, e.g., a second, gastrointestinal homing receptor, e.g., the primary T lymphocyte comprises CCR9 and integrin α4β7.

In one embodiment, the cell, e.g., the T lymphocyte, or a population of such cells, that comprises, e.g., has been engineered to express, a gastrointestinal homing receptor has been activated, expanded, or both activated and expanded in the presence of a Vitamin A metabolite. In specific embodiments, the Vitamin A metabolite is retinoic acid.

In certain embodiments, the homing receptor is a skin homing receptor. In a specific embodiment, the skin homing receptor is CLA (cutaneous lymphocyte-associated antigen receptor). In other specific embodiments, the skin homing receptor is CCR4, CCR8, or CCR10. In certain embodiments, the cell, e.g., a T lymphocyte, for example, a primary T lymphocyte, or population of such cells comprises, e.g., has been engineered to express, second skin homing receptor. In yet other embodiments, the cell, e.g., a T lymphocyte, for example, a primary T lymphocyte, or population of such cells, comprises, e.g., expresses, a third skin homing receptor. In yet other embodiments, the cell, e.g., a T lymphocyte, for example, a primary T lymphocyte, or population of such cells, comprises, e.g., expresses, a fourth skin homing receptor. In other embodiments, the cell or population of cells comprises, e.g., expresses, two or more skin homing receptors, e.g., two or more of CLA, CCR4, CCR8, and/or CCR10.

In one embodiment, the cell, e.g., the T lymphocyte, for example, the primary T lymphocyte, or population of such cells comprising, e.g., expressing, a skin-homing receptor has been activated, expanded, or both activated and expanded in the presence of a Vitamin D metabolite. In specific embodiments, the Vitamin D metabolite is 1,26-dihydroxycholecalciferol (1,25(OH)₂D₃). In some embodiments, the cell, e.g., the T lymphocyte, for example, the primary T lymphocyte, or population of such cells has been activated, expanded, or both activated and expanded in the presence of IL-12. In specific embodiments, the cell, e.g., the T lymphocyte, for example, the primary T lymphocyte, or population of such cells, have been activated, expanded, or activated and expanded in the presence of both IL-12 and a vitamin D metabolite.

In some embodiments, the cell, e.g., the T lymphocyte, for example, the primary T lymphocyte, or population of such cells, comprises, e.g., has been engineered to express, either a gastrointestinal homing receptor or a skin homing receptor, and additionally comprises a B cell homing receptor. In specific embodiments, the B cell homing receptor is CXCR5.

In another aspect, provided herein are methods of generating a cell, e.g., a T lymphocyte, for example, a primary T lymphocyte, or population of such cells, which expresses a homing receptor. In certain embodiments, provided herein is a method of generating a cell, e.g., a T lymphocyte, for example, a primary T lymphocyte, or population of such cells, that homes to the B cell zone of a lymph node, comprising engineering the cell or population of cells to express a B cell zone homing receptor in an amount or at a level sufficient to cause the cell or population of cells to home to the B cell zone of the lymph node. In a specific embodiment, the B cell zone homing receptor is CXCR5.

In certain embodiments, provided herein is a method of generating a cell, e.g., a T lymphocyte, for example, a primary T lymphocyte, or population of such cells, that homes to the gastrointestinal tract, comprising engineering the cell or population of cells to express a gastrointestinal homing receptor in an amount or at a level sufficient to cause the cell or population of cells to home to the gastrointestinal tract. In a specific embodiment, the gastrointestinal homing receptor is integrin α4β7. In other specific embodiments, the gastrointestinal homing receptor is CCR9. In certain embodiments, provided herein is a method of generating a cell, e.g., a T lymphocyte, for example, a primary T lymphocyte, or population of such cells, comprising engineering the cell or population of cells to express an additional gastrointestinal homing receptor, e.g., additionally express a second gastrointestinal homing receptor. In certain embodiments, the methods provided herein additionally comprise a step wherein the cell or population of cells is activated, expanded, or both activated and expanded in the presence of a Vitamin A metabolite. In specific embodiments, the Vitamin A metabolite is retinoic acid.

In certain embodiments, provided herein is a method of generating a cell, e.g., a T lymphocyte, for example, a primary T lymphocyte, or population of such cells, that homes to the skin, comprising engineering the cell or population of cells to express a skin homing receptor in an amount or at a level sufficient to cause the cell or population of cells to home to the skin. In a specific embodiment, the skin homing receptor is CLA. In other specific embodiments, the skin homing receptor is CCR4, CCR8, or CCR10. In certain embodiments, provided herein is a method of generating a cell, e.g., a T lymphocyte, for example, a primary T lymphocyte, or population of such cells, comprising engineering the cell or population of cells to express an additional skin homing receptor, e.g., additionally express a second skin homing receptor. In certain embodiments, provided herein is a method of generating a cell, e.g., a T lymphocyte, for example, a primary T lymphocyte, or population of such cells, comprising engineering the cell or population of cells to additionally express a third skin homing receptor. In certain embodiments, provided herein is a method of generating a cell, e.g., a T lymphocyte, for example, a primary T lymphocyte, or a population of such cells, comprising engineering the cell or population of cells to additionally express a fourth skin homing receptor. In certain embodiments, the methods provided herein additionally comprise a step wherein the cell or population of cells is activated, expanded, or both activated and expanded in the presence of a Vitamin D metabolite. In specific embodiments, the Vitamin D metabolite is 1,26-dihydroxycholecalciferol (1,25(OH)₂D₃). In certain embodiments, the methods provided herein additionally comprise a step wherein the cell or population of cells is activated, expanded, or both activated and expanded in the presence of IL-12.

In certain embodiments, provided herein is a method of generating a cell, e.g., a T lymphocyte, for example, a primary T lymphocyte, or a population of such cells, that is engineered to express a gastrointestinal homing receptor or a skin homing receptor, and additionally is engineered to express a B cell zone homing receptor. In specific embodiments, provided herein is a method of generating a cell, e.g., a T lymphocyte, for example, a primary T lymphocyte, or a population of such cells, that is engineered to express a gastrointestinal homing receptor or a skin homing receptor, and additionally is engineered to express CXCR5.

In certain embodiments, provided herein is a method of generating a cell, e.g., a T lymphocyte, for example, a primary T lymphocyte, or a population of such cells, that is engineered to express a gastrointestinal homing receptor or a skin homing receptor, which method comprises a step of administering to the T lymphocyte a lentiviral vector encoding a chimeric antigen receptor (CAR).

In another aspect, provided herein are methods of treating a cancer or tumor in an individual, comprising administering to an individual in need thereof a T lymphocyte, or a population of T lymphocytes, comprising (i) a B cell zone homing receptor; and (ii) a CAR. In certain embodiments, the B cell zone homing receptor is CXCR5.

In certain embodiments, provided herein are methods of treating a gastrointestinal cancer or tumor in an individual, comprising administering to an individual in need thereof a T lymphocyte, or a population of T lymphocytes, comprising, e.g., expressing, (i) a gastrointestinal homing receptor; and (ii) a CAR. In specific embodiments, the gastrointestinal homing receptor is integrin α4β7. In other specific embodiments, the gastrointestinal homing receptor is CCR9. In certain embodiments, provided herein are methods of treating a gastrointestinal cancer or tumor in an individual, comprising administering to an individual in need thereof a T lymphocyte, or a population of Ty lymphocytes, additionally comprising, e.g., expressing, a second gastrointestinal homing receptor.

In certain embodiments, provided herein are methods of treating a skin cancer or tumor in an individual, comprising administering to an individual in need thereof a T lymphocyte, or a population of T lymphocytes, comprising (i) a skin homing receptor; and (ii) a CAR. In specific embodiments, the skin homing receptor is CLA. In other specific embodiments, the skin homing receptor is CCR4, CCR8, or CCR10. In certain embodiments, provided herein are methods of treating a skin cancer or tumor in an individual, comprising administering to an individual in need thereof a T lymphocyte, or a population of T lymphocytes, additionally comprising a second skin homing receptor. In certain embodiments, provided herein are methods of treating a skin cancer or tumor in an individual, comprising administering to an individual in need thereof a T lymphocyte, or a population of T lymphocytes, additionally comprising a third skin homing receptor. In certain embodiments, provided herein are methods of treating a skin cancer or tumor in an individual, comprising administering to an individual in need thereof a T lymphocyte, or a population of T lymphocytes, additionally comprising a fourth skin homing receptor.

In certain embodiments, provided herein are methods of treating a gastrointestinal cancer or tumor in an individual, comprising administering to an individual in need thereof a T lymphocyte, or a population of T lymphocytes, comprising (i) a gastrointestinal homing receptor; and (ii) a CAR, wherein the T lymphocyte, or a population of T lymphocytes, further comprises a B cell zone homing receptor. In specific embodiments, the B cell zone homing receptor is CXCR5. In certain embodiments, the T lymphocytes provided herein are for use in methods of treating a gastrointestinal cancer or tumor.

In certain embodiments, provided herein are methods of treating a skin cancer or tumor in an individual, comprising administering to an individual in need thereof a T lymphocyte, or a population of T lymphocytes, comprising (i) a skin homing receptor; and (ii) a CAR, wherein the T lymphocyte, or a population of T lymphocytes, further comprises a B cell zone homing receptor. In specific embodiments, the B cell zone homing receptor is CXCR5. In certain embodiments, the T lymphocytes provided herein are for use in methods of treating a skin cancer or tumor.

In specific embodiments, provided herein are methods of treating a cancer or tumor in an individual, comprising administering to an individual in need thereof a T lymphocyte, or a population of T lymphocytes, comprising (i) a homing receptor; and (ii) a CAR, wherein the extracellular domain of the CAR binds an antigen selected from the group consisting of Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (melanoma antigen recognized by T lymphocytes; MART-1), myo-D₁, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, an abnormal p53 protein, fuc-GM1, GM2 (oncofetal antigen-immunogenic-1; OFA-I-1); GD2 (OFA-I-2), GM3, GD3, alpha-actinin-4, Bage-1, BCR-ABL, Bcr-Abl fusion protein, beta-catenin, CA 15-3 (CA 27.29\BCAA), CA 195, CA 242, CA-50, CAM43, Casp-8, cdc27, cdk4, cdkn2a, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, HER-2/neu, E2A-PRL, H4-RET, IGH-IGK, MYL-RAR, human papillomavirus (HPV) antigens E6 and E7, TSP-180, p185erbB2, p180erbB-3, c-met, nm-23H1, PSA, TAG-72-4, CA 19-9, CA 72-4, CAM 17.1, NuMa, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD22, CD27, CD30, CD70, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), integrin αvβ3 (CD61), galactin, and Ral-B. In specific embodiments, provided herein are methods of treating a gastrointestinal cancer or tumor in an individual, comprising administering to an individual in need thereof a T lymphocyte, or a population of T lymphocytes, comprising (i) a gastrointestinal homing receptor; and (ii) a CAR, wherein the extracellular domain of the CAR binds an antigen associated with, e.g., expressed by, a gastrointestinal tumor or cancer. In specific embodiments, the antigen associated with, e.g., expressed by, a gastrointestinal tumor or cancer is CEA, Her2, CA242, MUC1, CA125, or CA19-9. In specific embodiments, the T lymphocytes provided herein are for use in methods of treating a cancer or tumor.

In other specific embodiments, provided herein are methods of treating a skin cancer or tumor in an individual, comprising administering to an individual in need thereof a T lymphocyte, or a population of T lymphocytes, comprising (i) a skin homing receptor; and (ii) a CAR, wherein the extracellular domain of the CAR binds an antigen associated with a skin tumor or cancer. In specific embodiments, the antigen associated with, e.g., expressed by, a skin tumor or cancer is HMW-MAA, Her2, GD2, GD3, CEA, or SPAG9. In other specific embodiments, the T lymphocytes provided herein are for use in methods of treating a skin cancer or tumor.

### 4. DETAILED DESCRIPTION

Adaptive immune responses are initiated in secondary lymphoid organs, including the lymph nodes. B cells and T cells are sequestered in distinct regions of the lymph nodes, termed the “B cell zone,” located in the outer cortex of the lymph node, or follicles, and the “T cell zone,” which is more diffusely distributed in the area surrounding the follicles (also known as the paracortex) respectively. B cells and T cells express receptors that allow them to home to these respective zones so that they can be exposed to antigen. Intact antigens are present in the B cell zone, whereas in the T cell zone, antigens are presented by antigen-presenting cells, such as dendritic cells. Intact antigens, such as tumor antigens, are also present at the site of the tumor.

Provided herein are genetically modified cells, for example immune cells, such as T lymphocytes, e.g., human T lymphocytes, that comprise a receptor that causes a cell expressing said receptor to home to a particular anatomical zone, a particular tissue, or a particular type of cell, e.g., B cell zone of the lymph nodes, gastrointestinal tract, or skin. In a specific embodiment, the genetically modified cells provided herein are T lymphocytes. In certain embodiments, the genetically modified cells express a chimeric antigen receptor (CAR), as described in Section 4.4. In certain embodiments, the genetically modified cells express a CAR and endogenously express one or more homing receptors, e.g., receptors that that cause a cell expressing the receptors to home to the B cell zone of the lymph nodes, gastrointestinal tract, or skin. Without wishing to be bound by any particular mechanism or theory, it is thought that when the genetically modified cells herein express receptors that cause a cell expressing said receptors to home to a particular zone, they are more likely to be exposed to native antigen, where the cells, for example, cells expressing a CAR, are capable of being activated.

4.1 Cells Comprising B-Cell Zone Homing Receptors, Methods of Making, and Uses Thereof

In one embodiment, provided herein are genetically modified cells, for example immune cells, such as T lymphocytes, e.g., human T lymphocytes, that comprise, e.g., have been engineered to express, a receptor that causes the cells to home to the B cell zone of the lymph nodes, e.g., the follicles of the lymph node. Such a receptor is referred to herein as a “B cell zone homing receptor.” In specific embodiments, the B cell zone homing receptor is CXCR5, for example, human CXCR5. GenBank™ accession numbers NM_001716.4 and NM_032966.2 provide exemplary nucleotide sequences for human CXCR5. GenBank™ accession numbers NP_116743.1 and NP_001707.1 provide exemplary amino acid sequences for human CXCR5. Exemplary nucleotide and amino acid sequences for human homing receptors can be found in Table 1. In a specific embodiment, the genetically modified cells provided herein are T lymphocytes. In certain embodiments, the genetically modified cells express a B cell zone homing receptor and also express a CAR, as described in Section 4.4. In certain embodiments, the genetically modified cells express a CAR and endogenously express one or more B cell zone homing receptors.

Also provided herein is a method of generating genetically engineered T lymphocytes that home to the B cell zone of a lymph node, e.g., the follicles of a lymph node, comprising a step of engineering a T lymphocyte to express a B cell zone homing receptor, e.g., CXCR5, wherein said B cell zone homing receptor is expressed by the cell at a sufficient level or sufficient amount to cause the cell to home to the B cell zone of the lymph node. In some embodiments, the step of engineering a T cell to express a B cell zone homing receptor comprises a step of introducing to the cells one or more vectors comprising the receptor nucleic acid sequence(s), i.e., the nucleic acid sequence (s) encoding the receptor(s). In specific embodiments, the vector comprises the nucleic acid sequence for human CXCR5. In a certain embodiment, the step of engineering a T cell to express a B cell zone homing receptor is performed by any method known to one of skill in the art.

Exemplary nucleic acids useful for engineering a T lymphocyte to express a B cell zone homing receptor are disclosed, for example, in Section 4.5.

Also provided herein are methods of treating a cancer or tumor in an individual comprising administering to the individual a therapeutically effective amount of genetically modified cells, e.g. human T lymphocytes, that comprise (i) a receptor that causes a cell expressing said receptor to home to a B cell zone of the lymph nodes, e.g., the follicles of the lymph node, and (ii) a CAR. In specific embodiments, the B cell zone homing receptor is CXCR5. In certain embodiments, the genetically modified cells, e.g. human T lymphocytes, that comprise (i) a receptor that causes a cell expressing said receptor to home to a B cell zone of the lymph nodes, e.g., the follicles of the lymph node, and (ii) a CAR, suppress the proliferation of tumor cells. In certain embodiments, the genetically modified cells, e.g. human T lymphocytes, that comprise (i) a receptor that causes a cell expressing said receptor to home to a B cell zone of the lymph nodes, e.g., the follicles of the lymph node, and (ii) a CAR, inhibit growth of the tumor. In certain embodiments, the genetically modified cells, e.g. human T lymphocytes, that comprise (i) a receptor that causes a cell expressing said receptor to home to a B cell zone of the lymph nodes, e.g., the follicles of the lymph node, and (ii) a CAR, kill tumor cells. In specific embodiments, the extracellular domain of the CAR binds an antigen binds an antigen selected from the group consisting of Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, an abnormal p53 protein, fuc-GM1, GM2 (oncofetal antigen-immunogenic-1; OFA-I-1); GD2 (OFA-I-2), GM3, GD3, alpha-actinin-4, Bage-1, BCR-ABL, Bcr-Abl fusion protein, beta-catenin, CA 15-3 (CA 27.29\BCAA), CA 195, CA 242, CA-50, CAM43, Casp-8, cdc27, cdk4, cdkn2a, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, HER-2/neu, E2A-PRL, H4-RET, IGH-IGK, MYL-RAR, human papillomavirus (HPV) antigens E6 and E7, TSP-180, p185erbB2, p180erbB-3, c-met, nm-23H1, PSA, TAG-72-4, CA 19-9, CA 72-4, CAM 17.1, NuMa, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD22, CD27, CD30, CD70, EGFRvIII (epidermal growth factor variant III), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), integrin αvβ3 (CD61), galactin, and Ral-B. Also provided herein are genetically modified cells for use in methods of treating a cancer or tumor.

4.2. Cells Comprising Gastrointestinal Homing Receptors, Methods of Making, and Uses Thereof

In one embodiment, provided herein are genetically modified cells, for example immune cells, such as T lymphocytes, e.g., human T lymphocytes, that comprise, e.g., have been engineered to express, a receptor that causes a cell expressing said receptor to home to the gastrointestinal tract, e.g., gastrointestinal organs, tissues, or cells. Such a receptor that causes a cell to home to the gastrointestinal tract is referred to herein as a “gastrointestinal homing receptor.” In a specific embodiment, the genetically modified cells provided herein are T lymphocytes. In certain embodiments, the gastrointestinal homing receptor is CCR9 or integrin α4β7, for example, human CCR9 or human integrin α4β7. GenBank™ accession numbers NM_031200.2 and NM001256369.1 provide exemplary nucleotide sequences for human CCR9. GenBank™ accession numbers NP_112477.1 and NP_001243298.1 provide exemplary amino acid sequences for human CCR9. GenBank™ accession numbers NM_000885.4 and NM_000889.2 provide exemplary nucleotide sequences for human α4 and human β7, respectively. GenBank™ accession numbers NP_000876.3 and NP_000880.1 provide exemplary amino acid sequences for human α4 and human β7, respectively. Exemplary nucleotide and amino acid sequences for human homing receptors can be found in Table 1. In some embodiments, the genetically modified cells further comprise a second gastrointestinal homing receptor. In some embodiments, the genetically modified cells comprise a first gastrointestinal homing receptor, wherein the first gastrointestinal homing receptor is CCR9, and further comprise a second gastrointestinal homing receptor, wherein the second gastrointestinal homing receptor is integrin α4β7. In other specific embodiments, the genetically modified cells comprise the gastrointestinal-homing receptor CXCR3.

In certain embodiments, the genetically modified cells containing one or more gastrointestinal homing receptors are expanded, activated, or both expanded and activated in the presence of a Vitamin A metabolite. In specific embodiments, the expansion, activation, or both expansion and activation occurs in vivo, in vitro, or ex vivo. In specific embodiments, the Vitamin A metabolite is retinoic acid. In certain embodiments, the genetically modified cells containing one or more gastrointestinal homing receptors additionally comprise a B cell zone homing receptor. In specific embodiments, the B cell zone homing receptor is CXCR5. In certain embodiments, the genetically modified cells express a gastrointestinal homing receptor and also express a CAR, as described in Section 4.4. In certain embodiments, the genetically modified cells express a CAR and endogenously express one or more gastrointestinal homing receptors.

Also provided herein are methods of generating genetically modified cells, e.g. human T lymphocytes, which comprise one or more receptors that cause a cell expressing the one or more receptors to home to the gastrointestinal tract, e.g., gastrointestinal organs, skin, or tissue. In certain embodiments, T lymphocytes comprising one or more receptors that that cause a cell expressing the one or more receptors to home to the gastrointestinal tract, e.g., CCR9 or integrin α4β7, are generated by a method comprising a step of engineering a T lymphocyte to express one or more gastrointestinal homing receptors. In some embodiments, the step of engineering a T cell to express one or more gastrointestinal homing receptors comprises introducing to the cells one or more vectors comprising a nucleic acid sequence encoding the receptor. In specific embodiments, the vector comprises the nucleic acid sequence for human CCR9, the nucleic acid sequence for human integrin α4β7, or both.

In certain embodiments, T lymphocytes that home to the gastrointestinal tract are generated by a method comprising a step of treating the cells with a molecule that induces the expression of one or more gastrointestinal homing receptors, e.g., CCR9 or α4β7. In specific embodiments, the molecule is Vitamin A.

In certain embodiments, the method for generating the genetically modified T lymphocytes that comprise one or more receptors that that cause a cell expressing the one or more receptors to home to the gastrointestinal tract comprises a step of expanding the cells, which step is carried out in the presence of a vitamin A metabolite. In certain embodiments, the method for generating the genetically modified T lymphocytes that comprise one or more receptors homing to the gastrointestinal tract comprises a step of activating the cells, which step is carried out in the presence of a vitamin A metabolite. In certain embodiments, both the expanding and activating steps are carried out in the presence of a vitamin A metabolite. In certain embodiments the vitamin A metabolite is retinoic acid. In a certain embodiment, the step of engineering a T cell to express a gastrointestinal homing receptor is performed by any method known to one of skill in the art.

Exemplary nucleic acids useful for engineering a T lymphocyte to express a gastrointestinal homing receptor are discussed, for example, in Section 4.5.

Also provided herein are methods of treating a gastrointestinal cancer or tumor in an individual comprising administering to the individual a therapeutically effective amount of genetically modified cells, e.g. human T lymphocytes that comprise (i) one or more receptors that cause a cell expressing the one or more receptors to home to the gastrointestinal tract, and (ii) a CAR. In certain embodiments, the gastrointestinal homing receptor is CCR9 or α4β7. In some embodiments, the genetically modified cells further comprise a second gastrointestinal homing receptor. In some embodiments, the genetically modified cells comprise a first gastrointestinal homing receptor, wherein the first gastrointestinal homing receptor is CCR9, and further comprise a second gastrointestinal homing receptor, wherein the second gastrointestinal homing receptor is α4β7. In certain embodiments, the genetically modified cells, e.g. human T lymphocytes, that comprise (i) one or more receptors that cause a cell expressing the one or more receptors to home to the gastrointestinal tract, and (ii) a CAR, suppress the proliferation of tumor cells. In certain embodiments, the genetically modified cells, e.g. human T lymphocytes, that comprise (i) one or more receptors that cause a cell expressing the one or more receptors to home to the gastrointestinal tract, and (ii) a CAR, inhibit growth of the tumor. In certain embodiments, the genetically modified cells, e.g. human T lymphocytes, that comprise (i) one or more receptors that cause a cell expressing the one or more receptors to home to the gastrointestinal tract, and (ii) a CAR, kill tumor cells. In specific embodiments, the gastrointestinal cancer or tumor is liver cancer, stomach cancer, esophageal cancer, gallbladder cancer, colorectal cancer, anal cancer, or pancreatic cancer. In certain embodiments, the extracellular domain of the CAR binds an antigen associated with a gastrointestinal cancer or tumor. Also provided herein are genetically modified cells for use in methods of treating a gastrointestinal cancer or tumor.

4.3. Cells Comprising Skin Homing Receptors, Methods of Making, and Uses Thereof

In one embodiment, provided herein are genetically modified cells, for example immune cells, such as T lymphocytes, e.g., human T lymphocytes, that comprise a receptor that causes a cell expressing said receptor to home to the skin, e.g., skin tissue, or skin cells. In a specific embodiment, the genetically modified cells provided herein are T lymphocytes. In certain embodiments, the skin homing receptor is CCR10, CCR8, CCR4, or CLA, for example, human CCR10, human CCR8, human CCR4, or human CLA. GenBank™ accession numbers NM_016602.2 and AF215981.1 provide exemplary nucleotide sequences for human CCR10. GenBank™ accession numbers NP_057686.2 and P46092.3 provide exemplary amino acid sequences for human CCR10. GenBank™ accession numbers NM_005201.3 and BC107159.1 provide exemplary nucleotide sequences for human CCR8. GenBank™ accession numbers NP_005192.1 and AAI07160.1 provide exemplary amino acid sequences for human CCR8. GenBank™ accession number NM_005508.4 provides an exemplary nucleotide sequence for human CCR4. GenBank™ accession number P51679.1 provides an exemplary amino acid sequence for human CCR4. GenBank™ accession numbers NM_001206609.1 and NM_003006.4 provide exemplary nucleotide sequences for human CLA. GenBank™ accession numbers NP_001193538.1 and NP_002997.2 provide exemplary amino acid sequences for human CLA. Exemplary nucleotide and amino acid sequences for human homing receptors can be found in Table 1. In some embodiments, the genetically modified cells further comprise a second skin homing receptor. In some embodiments, the genetically modified cells comprise a first skin homing receptor, wherein the first skin homing receptor is CCR10, and further comprise a second skin homing receptor, wherein the second skin homing receptor is CLA. In some embodiments, the genetically modified cells comprise a first skin homing receptor, wherein the first skin homing receptor is CCR10, and further comprise a second skin homing receptor, wherein the second skin homing receptor is CCR4. In some embodiments, the genetically modified cells comprise a first skin homing receptor, wherein the first skin homing receptor is CCR4, and further comprise a second skin homing receptor, wherein the second skin homing receptor is CLA. In some embodiments, the genetically modified cells further comprise a third skin homing receptor. In some embodiments, the genetically modified cells comprise a first skin homing receptor, wherein the first skin homing receptor is CCR10, further comprise a second skin homing receptor, wherein the second skin homing receptor is CCR4, and further comprise a third skin homing receptor, wherein the third skin homing receptor is CLA. In some embodiments, the genetically modified cells comprise a first skin homing receptor, wherein the first skin homing receptor is CCR8, and further comprise a second skin homing receptor, wherein the second skin homing receptor is CLA, CCR4, or CCR10. In some embodiments, the genetically modified cells comprise a first skin homing receptor, wherein the first skin homing receptor is CCR8, further comprise a second skin homing receptor, wherein the second skin homing receptor is CLA, CCR4, or CCR10, and further comprise a third skin homing receptor, wherein the third skin homing receptor is distinct from the second skin homing receptor, and is selected from the group consisting of CLA, CCR4, and CCR10. In some embodiments, the genetically modified cells further comprise a third skin homing receptor. In some embodiments, the genetically modified cells comprise a first skin homing receptor, wherein the first skin homing receptor is CCR10, further comprise a second skin homing receptor, wherein the second skin homing receptor is CCR4, further comprise a third skin homing receptor, wherein the third skin homing receptor is CLA, and further comprise a fourth skin homing receptor, wherein the fourth skin homing receptor is CCR8. In certain embodiments, the genetically modified cells comprise one or more skin homing receptors. In other specific embodiments, the genetically modified cells comprise the skin-homing receptor CCR6.

In certain embodiments, the genetically modified cells containing one or more skin homing receptors are expanded, activated, or both expanded and activated in the presence of a Vitamin D metabolite. In specific embodiments, the expansion, activation, or both expansion and activation occurs in vivo, in vitro, or ex vivo. In specific embodiments, the Vitamin D metabolite is 1,25-dihydroxycholecalciferol (1,25(OH)₂D₃). In certain embodiments, the genetically modified cells containing one or more skin homing receptors are expanded, activated, or both expanded and activated in the presence of IL-12. In specific embodiments, the expansion, activation, or both expansion and activation occurs in vivo, in vitro, or ex vivo. In more specific embodiments, the genetically modified cells containing one or more skin homing receptors are expanded, activated, or both expanded and activated in the presence of a Vitamin D metabolite and IL-12. In specific embodiments, the expansion, activation, or both expansion and activation occurs in vivo, in vitro, or ex vivo. In certain embodiments, the genetically modified cells containing one or more skin homing receptors additionally comprise a B cell zone homing receptor. In specific embodiments, the B cell zone homing receptor is CXCR5. In certain embodiments, the genetically modified cells express a skin homing receptor and also express a CAR, as described in Section 4.4. In certain embodiments, the genetically modified cells express a CAR and endogenously express one or more skin homing receptors.

Also provided herein are methods of generating genetically modified cells, e.g. human T lymphocytes, that comprise one or more receptors homing to the skin, e.g., skin tissue or cells. In certain embodiments, T lymphocytes that home to the skin are generated by a method comprising a step of engineering the T lymphocytes to express a skin homing receptor, e.g., CCR4, CCR8, CCR10, or CLA. In some embodiments, the step of engineering the T lymphocytes to express a skin homing receptor comprises introducing into the cells one or more vectors comprising the receptor nucleic acid sequence(s), i.e., the nucleic acid sequence(s) encoding the receptor(s). In specific embodiments, the vector comprises the nucleic acid sequence for human CCR10, the nucleic acid sequence for human CLA, or both. In specific embodiments, the vector comprises the nucleic acid sequence for human CCR4, and optionally the nucleic acid sequence for human CLA. In specific embodiments, the vector comprises the nucleic acid sequence for human CCR4 and the nucleic acid sequence for human CCR10. In specific embodiments, the vector comprises the nucleic acid sequence for human CCR10, the nucleic acid sequence for human CCR4, and the nucleic acid sequence for human CLA. In specific embodiments, the vector comprises the nucleic acid sequence for human CCR8. In specific embodiments, the vector comprises the nucleic acid sequence for human CCR8, and optionally the nucleic acid sequence for human CLA. In specific embodiments, the vector comprises the nucleic acid sequence for human CCR8 and the nucleic acid sequence for human CCR10. In specific embodiments, the vector comprises the nucleic acid sequence for human CCR8, the nucleic acid sequence for human CCR4, and the nucleic acid sequence for human CLA. In specific embodiments, the vector comprises the nucleic acid sequence for human CCR8, the nucleic acid sequence for human CCR10, and the nucleic acid sequence for human CLA. In specific embodiments, the vector comprises the nucleic acid sequence for human CCR8, the nucleic acid sequence for human CCR4, and the nucleic acid sequence for human CCR10. In specific embodiments, the vector comprises the nucleic acid sequence for human CCR8, the nucleic acid sequence for human CCR4, the nucleic acid for CCR10, and the nucleic acid sequence for human CLA.

In certain embodiments, cells, e.g., T lymphocytes, that home to the skin are generated by a method comprising a step of treating the cells, e.g., T lymphocytes, with a molecule that induces, e.g., increases, the expression of one or more skin homing receptors, e.g., CCR4, CCR10, CCR8, or CLA. In specific embodiments, the molecule is Vitamin D. In certain embodiments, the induction of expression of skin homing receptors is aided by treating the cells, e.g., T lymphocytes, with IL-12, e.g., contacting the cells with IL-12 in an amount and for a time sufficient to increase expression of one or more of CCR4, CCR8, CCR10, or CLA by said cells.

In certain embodiments, the method for generating the genetically modified T lymphocytes that comprise one or more receptors that cause a cell expressing the one or more receptors to home to the skin, comprises a step of expanding the cells, which step is carried out in the presence of a vitamin D metabolite and, optionally, IL-12. In certain embodiments, the method for generating the genetically modified T lymphocytes that comprise one or more receptors that that cause a cell expressing the one or more receptors to home to the gastrointestinal tract, comprises a step of activating the cells, which step is carried out in the presence of a vitamin D metabolite, and, optionally, IL-12. In certain embodiments, both the expanding and activating steps are carried out in the presence of a vitamin D metabolite, and, optionally, IL-12. In certain embodiments the vitamin D metabolite is 1,25(OH)₂D₃. In a certain embodiment, the step of engineering a T cell to express a skin homing receptor is performed by any method known to one of skill in the art.

Exemplary nucleic acids useful for engineering a T lymphocyte to express a skin homing receptor are discussed, for example, in Section 4.5.

Also provided herein are methods of treating a skin cancer or tumor in an individual comprising administering to the individual a therapeutically effective amount of genetically modified cells, e.g. human T lymphocytes that comprise (i) one or more receptors that that cause a cell expressing the one or more receptors to home to the skin, and (ii) a CAR. In certain embodiments, the skin homing receptor is CCR10, CCR8, CCR4, or CLA. In some embodiments, the genetically modified cells further comprise a second skin homing receptor. In some embodiments, the genetically modified cells comprise a first skin homing receptor, wherein the first skin homing receptor is CCR10, and further comprise a second skin homing receptor, wherein the second skin homing receptor is CLA. In some embodiments, the genetically modified cells comprise a first skin homing receptor, wherein the first skin homing receptor is CCR10, and further comprise a second skin homing receptor, wherein the second skin homing receptor is CCR4. In some embodiments, the genetically modified cells comprise a first skin homing receptor, wherein the first skin homing receptor is CCR10, and further comprise a second skin homing receptor, wherein the second skin homing receptor is CCR8. In some embodiments, the genetically modified cells comprise a first skin homing receptor, wherein the first skin homing receptor is CCR4, and further comprise a second skin homing receptor, wherein the second skin homing receptor is CLA. In some embodiments, the genetically modified cells comprise a first skin homing receptor, wherein the first skin homing receptor is CCR4, and further comprise a second skin homing receptor, wherein the second skin homing receptor is CCR8. In some embodiments, the genetically modified cells comprise a first skin homing receptor, wherein the first skin homing receptor is CLA, and further comprise a second skin homing receptor, wherein the second skin homing receptor is CCR8. In some embodiments, the genetically modified cells further comprise a third skin homing receptor. In some embodiments, the genetically modified cells comprise a first skin homing receptor, wherein the first skin homing receptor is CCR10, further comprise a second skin homing receptor, wherein the second skin homing receptor is CCR4, and further comprise a third skin homing receptor, wherein the third skin homing receptor is CLA. In some embodiments, the genetically modified cells comprise a first skin homing receptor, wherein the first skin homing receptor is CCR10, further comprise a second skin homing receptor, wherein the second skin homing receptor is CCR4, and further comprise a third skin homing receptor, wherein the third skin homing receptor is CCR8. In some embodiments, the genetically modified cells comprise a first skin homing receptor, wherein the first skin homing receptor is CCR8, further comprise a second skin homing receptor, wherein the second skin homing receptor is CCR4, and further comprise a third skin homing receptor, wherein the third skin homing receptor is CLA. In some embodiments, the genetically modified cells comprise a first skin homing receptor, wherein the first skin homing receptor is CCR10, further comprise a second skin homing receptor, wherein the second skin homing receptor is CCR8, and further comprise a third skin homing receptor, wherein the third skin homing receptor is CLA. In some embodiments, the genetically modified cells further comprise a fourth skin homing receptor. In some embodiments, the genetically modified cells comprise a first skin homing receptor, wherein the first skin homing receptor is CCR10, further comprise a second skin homing receptor, wherein the second skin homing receptor is CCR4, further comprise a third skin homing receptor, wherein the third skin homing receptor is CLA, and further comprise a fourth skin homing receptor, wherein the fourth skin homing receptor is CCR8. In certain embodiments, the genetically modified cells, e.g. human T lymphocytes, that comprise (i) one or more receptors that cause a cell expressing the one or more receptors to home to the skin, and (ii) a CAR, suppress the proliferation of tumor cells. In certain embodiments, the genetically modified cells, e.g. human T lymphocytes, that comprise (i) one or more receptors that cause a cell expressing the one or more receptors to home to the skin, and (ii) a CAR, inhibit growth of the tumor. In certain embodiments, the genetically modified cells, e.g. human T lymphocytes, that comprise (i) one or more receptors that cause a cell expressing the one or more receptors to home to the skin, and (ii) a CAR, kill tumor cells. In specific embodiments, the skin cancer or tumor is melanoma, squamous cell carcinoma, or basal cell carcinoma. In certain embodiments, the extracellular domain of the CAR binds an antigen associated with a skin cancer or tumor. Also provided herein are genetically modified cells for use in methods of treating a skin cancer or tumor.

TABLE 1
Exemplary nucleotide and amino acid sequences for human homing receptors.

| | GenBank | | |
|---|---|---|---|
| SEQ | Accession | | |
| ID | Number and | | |
| NO: | Description | Sequence | |
| 1 | NM_001716.4 | 1 | aaaaaaaaaa agtgatgagt tgtgaggcag gtcgcggccc tactgcctca ggagacgatg |
| | Exemplary nucleic | 61 | cgcagctcat ttgcttaaat ttgcagctga cggctgccac ctctctagag gcacctggcg |
| | acid sequence | 121 | gggagcctct caacataaga cagtgaccag tctggtgact cacagccggc acagccatga |
| | encoding human | 181 | actacccgct aacgctggaa atggacctcg agaacctgga ggacctgttc tgggaactgg |
| | CXCR5 | 241 | acagattgga caactataac gacacctccc tggtggaaaa tcatctctgc cctgccacag |
| | | 301 | aggggcccct catggcctcc ttcaaggccg tgttcgtgcc cgtggcctac agcctcatct |
| | | 361 | tcctcctggg cgtgatcggc aacgtcctgg tgctggtgat cctggagcgg caccggcaga |
| | | 421 | cacgcagttc cacggagacc ttcctgttcc acctggccgt ggccgacctc ctgctggtct |
| | | 481 | tcatcttgcc ctttgccgtg gccgagggct ctgtgggctg ggtcctgggg accttcctct |
| | | 541 | gcaaaactgt gattgccctg cacaaagtca acttctactg cagcagcctg ctcctggcct |
| | | 601 | gcatcgccgt ggaccgctac ctggccattg tccacgccgt ccatgcctac cgccaccgcc |
| | | 661 | gcctcctctc catccacatc acctgtggga ccatctggct ggtgggcttc ctccttgcct |
| | | 721 | tgccagagat tctcttcgcc aaagtcagcc aaggccatca caacaactcc ctgccacgtt |
| | | 781 | gcaccttctc ccaagagaac caagcagaaa cgcatgcctg gttcacctcc cgattcctct |
| | | 841 | accatgtggc gggattcctg ctgcccatgc tggtgatggg ctggtgctac gtgggggtag |
| | | 901 | tgcacaggtt gcgccaggcc cagcggcgcc ctcagcggca gaaggcagtc agggtggcca |
| | | 961 | tcctggtgac aagcatcttc ttcctctgct ggtcacccta ccacatcgtc atcttcctgg |
| | | 1021 | acaccctggc gaggctgaag gccgtggaca atacctgcaa gctgaatggc tctctccccg |
| | | 1081 | tggccatcac catgtgtgag ttcctgggcc tggcccactg ctgcctcaac cccatgctct |
| | | 1141 | acactttcgc cggcgtgaag ttccgcagtg acctgtcgcg gctcctgacg aagctgggct |
| | | 1201 | gtaccggccc tgcctccctg tgccagctct tccctagctg gcgcaggagc agtctctctg |
| | | 1261 | agtcagagaa tgccacctct ctcaccacgt tctaggtccc agtgtcccct tttattgctg |
| | | 1321 | cttttccttg gggcaggcag tgatgctgga tgctccttcc aacaggagct gggatcctaa |
| | | 1381 | gggctcaccg tggctaagag tgtcctagga gtatcctcat ttggggtagc tagaggaacc |
| | | 1441 | aacccccatt tctagaacat ccctgccagc tcttctgccg gccctggggc taggctggag |
| | | 1501 | cccagggagc ggaaagcagc tcaaaggcac agtgaaggct gtccttaccc atctgcaccc |
| | | 1561 | ccctgggctg agagaacctc acgcacctcc catcctaatc atccaatgct caagaaacaa |
| | | 1621 | cttctacttc tgcccttgcc aacggagagc gcctgcccct cccagaacac actccatcag |
| | | 1681 | cttaggggct gctgacctcc acagcttccc ctctctcctc ctgcccacct gtcaaacaaa |
| | | 1741 | gccagaagct gagcaccagg ggatgagtgg aggttaaggc tgaggaaagg ccagctggca |
| | | 1801 | gcagagtgtg gccttcggac aactcagtcc ctaaaaacac agacattctg ccaggccccc |
| | | 1861 | aagcctgcag tcatcttgac caagcaggaa gctcagactg gttgagttca ggtagctgcc |
| | | 1921 | cctggctctg accgaaacag cgctgggtcc accccatgtc accggatcct gggtggtctg |
| | | 1981 | caggcagggc tgactctagg tgcccttgga ggccagccag tgacctgagg aagcgtgaag |
| | | 2041 | gccgagaagc aagaaagaaa cccgacagag ggaagaaaag agctttcttc ccgaacccca |
| | | 2101 | aggagggaga tggatcaatc aaacccggcg gtcccctccg ccaggcgaga tggggtgggg |
| | | 2161 | tggagaactc ctagggtggc tgggtccagg ggatgggagg ttgtgggcat tgatggggaa |
| | | 2221 | ggaggctggc ttgtcccctc ctcactccct tcccataagc tatagacccg aggaaactca |
| | | 2281 | gagtcggaac ggagaaaggt ggactggaag gggcccgtgg gagtcatctc aaccatcccc |
| | | 2341 | tccgtggcat caccttaggc agggaagtgt aagaaacaca ctgaggcagg gaagtcccca |
| | | 2401 | ggccccagga agccgtgccc tgcccccgtg aggatgtcac tcagatggaa ccgcaggaag |
| | | 2461 | ctgctccgtg cttgtttgct cacctggggt gtgggaggcc cgtccggcag ttctgggtgc |
| | | 2521 | tccctaccac ctccccagcc tttgatcagg tggggagtca gggacccctg cccttgtccc |
| | | 2581 | actcaagcca agcagccaag ctccttggga ggccccactg gggaaataac agctgtggct |
| | | 2641 | cacgtgagag tgtcttcacg gcaggacaac gaggaagccc taagacgtcc cttttttctc |
| | | 2701 | tgagtatctc ctcgcaagct gggtaatcga tgggggagtc tgaagcagat gcaaagaggc |
| | | 2761 | aagaggctgg attttgaatt ttctttttaa taaaaaggca cctataaaac aggtcaatac |
| | | 2821 | agtacaggca gcacagagac ccccggaaca agcctaaaaa ttgtttcaaa ataaaaacca |
| | | 2881 | agaagatgtc ttcacatatt gtaaaaaaaa aaaaaaaaa |
| 2 | NM_0329662 | 1 | ccactctaag gaatgcggtc cctttgacag gcgaaaaact gaagttggaa aagacaaagt |
| | Exemplary nucleic | 61 | gatttgttca aaattgaaat ttgaaacttg acatttggtc agtgggccct atgtaggaaa |
| | acid sequence | 121 | aaacctccaa gagagctagg gttcctctca gagaggaaag acaggtcctt aggtcctcac |
| | encoding human | 181 | cctcccgtct ccttgccctt gcagttctgg gaactggaca gattggacaa ctataacgac |
| | CNCR5 | 241 | acctccctgg tggaaaatca tctctgccct gccacagagg ggcccctcat ggcctccttc |
| | | 301 | aaggccgtgt tcgtgcccgt ggcctacagc ctcatcttcc tcctgggcgt gatcggcaac |
| | | 361 | gtcctggtgc tggtgatcct ggagcggcac cggcagacac gcagttccac ggagaccttc |
| | | 421 | ctgttccacc tggccgtggc cgacctcctg ctggtcttca tcttgccctt tgccgtggcc |
| | | 481 | gagggctctg tgggctgggt cctggggacc ttcctctgca aaactgtgat tgccctgcac |
| | | 541 | aaagtcaact tctactgcag cagcctgctc ctggcctgca tcgccgtgga ccgctacctg |
| | | 601 | gccattgtcc acgccgtcca tgcctaccgc caccgccgcc tcctctccat ccacatcacc |
| | | 661 | tgtgggacca tctggctggt gggcttcctc cttgccttgc cagagattct cttcgccaaa |
| | | 721 | gtcagccaag gccatcacaa caactccctg ccacgttgca ccttctccca agagaaccaa |
| | | 781 | gcagaaacgc atgcctggtt cacctcccga ttcctctacc atgtggcggg attcctgctg |
| | | 841 | cccatgctgg tgatgggctg gtgctacgtg ggggtagtgc acaggttgcg ccaggcccag |
| | | 901 | cggcgccctc agcggcagaa ggcagtcagg gtggccatcc tggtgacaag catcttcttc |
| | | 961 | ctctgctggt caccctacca catcgtcatc ttcctggaca ccctggcgag gctgaaggcc |
| | | 1021 | gtggacaata cctgcaagct gaatggctct ctccccgtgg ccatcaccat gtgtgagttc |
| | | 1081 | ctgggcctgg cccactgctg cctcaacccc atgctctaca ctttcgccgg cgtgaagttc |
| | | 1141 | cgcagtgacc tgtcgcggct cctgacgaag ctgggctgta ccggccctgc ctccctgtgc |
| | | 1201 | cagctcttcc ctagctggcg caggagcagt ctctctgagt cagagaatgc cacctctctc |
| | | 1261 | accacgttct aggtcccagt gtcccctttt attgctgctt ttccttgggg caggcagtga |
| | | 1321 | tgctggatgc tccttccaac aggagctggg atcctaaggg ctcaccgtgg ctaagagtgt |
| | | 1381 | cctaggagta tcctcatttg gggtagctag aggaaccaac ccccatttct agaacatccc |
| | | 1441 | tgccagctct tctgccggcc ctggggctag gctggagccc agggagcgga aagcagctca |
| | | 1501 | aaggcacagt gaaggctgtc cttacccatc tgcacccccc tgggctgaga gaacctcacg |
| | | 1561 | cacctcccat cctaatcatc caatgctcaa gaaacaactt ctacttctgc ccttgccaac |
| | | 1621 | ggagagcgcc tgcccctccc agaacacact ccatcagctt aggggctgct gacctccaca |
| | | 1681 | gcttcccctc tctcctcctg cccacctgtc aaacaaagcc agaagctgag caccagggga |
| | | 1741 | tgagtggagg ttaaggctga ggaaaggcca gctggcagca gagtgtggcc ttcggacaac |
| | | 1801 | tcagtcccta aaaacacaga cattctgcca ggcccccaag cctgcagtca tcttgaccaa |
| | | 1861 | gcaggaagct cagactggtt gagttcaggt agctgcccct ggctctgacc gaaacagcgc |
| | | 1921 | tgggtccacc ccatgtcacc ggatcctggg tggtctgcag gcagggctga ctctaggtgc |
| | | 1981 | ccttggaggc cagccagtga cctgaggaag cgtgaaggcc gagaagcaag aaagaaaccc |
| | | 2041 | gacagaggga agaaaagagc tttcttcccg aaccccaagg agggagatgg atcaatcaaa |
| | | 2101 | cccggcggtc ccctccgcca ggcgagatgg ggtggggtgg agaactccta gggtggctgg |
| | | 2161 | gtccagggga tgggaggttg tgggcattga tggggaagga ggctggcttg tcccctcctc |
| | | 2221 | actcccttcc cataagctat agacccgagg aaactcagag tcggaacgga gaaaggtgga |
| | | 2281 | ctggaagggg cccgtgggag tcatctcaac catcccctcc gtggcatcac cttaggcagg |
| | | 2341 | gaagtgtaag aaacacactg aggcagggaa gtccccaggc cccaggaagc cgtgccctgc |
| | | 2401 | ccccgtgagg atgtcactca gatggaaccg caggaagctg ctccgtgctt gtttgctcac |
| | | 2461 | ctggggtgtg ggaggcccgt ccggcagttc tgggtgctcc ctaccacctc cccagccttt |
| | | 2521 | gatcaggtgg ggagtcaggg acccctgccc ttgtcccact caagccaagc agccaagctc |
| | | 2581 | cttgggaggc cccactgggg aaataacagc tgtggctcac gtgagagtgt cttcacggca |
| | | 2641 | ggacaacgag gaagccctaa gacgtccctt ttttctctga gtatctcctc gcaagctggg |
| | | 2701 | taatcgatgg gggagtctga agcagatgca aagaggcaag aggctggatt ttgaattttc |
| | | 2761 | tttttaataa aaaggcacct ataaaacagg tcaatacagt acaggcagca cagagacccc |
| | | 2821 | cggaacaagc ctaaaaattg tttcaaaata aaaaccaaga agatgtcttc acatattgta |
| | | 2881 | aaaaaaaaaa aaaaaa |
| 3 | NP_116743.1 | 1 | masfkavfvp vayslifllg vignvlvlvi lerhrqtrss tetflfhlav adlllvfilp |
| | Exemplary amino | 61 | favaegsvgw vlgtflcktv ialhkvnfyc sslllaciav drylaivhav hayrhrrlls |
| | acid sequence for | 121 | ihitcgtiwl vgfllalpei lfakvsqghh nnslprctfs qenqaethaw ftsrflyhva |
| | human CXCR5 | 181 | gfllpmlvmg wcyvgvvhrl rqaqrrpqrq kavrvailvt sifflcwspy hivifldtla |
| | precursor | 241 | rlkavdntck lngslpvait mceflglahc clnpmlytfa gvkfrsdlsr lltklgctgp |
| | | 301 | aslcqlfpsw rrsslsesen atslttf |
| 4 | NP_001707.1 | 1 | mnypltlemd lenledlfwe ldrldnyndt slvenhlcpa tegplmasfk avfvpvaysl |
| | Exemplary amino | 61 | ifllgvignv lvlvilerhr qtrsstetfl fhlavadlll vfilpfavae gsvgwvlgtf |
| | acid sequence for | 121 | lcktvialhk vnfycsslll aciavdryla ivhavhayrh rrllsihitc gtiwlvgfll |
| | human CXCR5 | 181 | alpeilfakv sqghhnnslp rctfsqenqa ethawftsrf lyhvagfllp mlvmgwcyvg |
| | precursor | 241 | vvhrlrqaqr rpqrqkavrv ailvtsiffl cwspyhivif ldtlarlkav dntcklngsl |
| | | 301 | pvaitmcefl glahcclnpm lytfagvkfr sdlsrlltkl gctgpaslcq lfpswrrssl |
| | | 361 | sesenatslt tf |
| 5 | NM_031200.2 | 1 | gcttcctttc tcgtgttgtt atcgggtagc tgcctgctca gaacccacaa agcctgcccc |
| | Exemplary nucleic | 61 | tcatcccagg cagagagcaa cccagctctt tccccagaca ctgagagctg gtggtgcctg |
| | acid sequence | 121 | ctgtcccagg gagagttgca tcgccctcca cagagcaggc ttgcatctga ctgacccacc |
| | encoding human | 181 | atgacaccca cagacttcac aagccctatt cctaacatgg ctgatgacta tggctctgaa |
| | CCR9 | 241 | tccacatctt ccatggaaga ctacgttaac ttcaacttca ctgacttcta ctgtgagaaa |
| | | 301 | aacaatgtca ggcagtttgc gagccatttc ctcccaccct tgtactggct cgtgttcatc |
| | | 361 | gtgggtgcct tgggcaacag tcttgttatc cttgtctact ggtactgcac aagagtgaag |
| | | 421 | accatgaccg acatgttcct tttgaatttg gcaattgctg acctcctctt tcttgtcact |
| | | 481 | cttcccttct gggccattgc tgctgctgac cagtggaagt tccagacctt catgtgcaag |
| | | 541 | gtggtcaaca gcatgtacaa gatgaacttc tacagctgtg tgttgctgat catgtgcatc |
| | | 601 | agcgtggaca ggtacattgc cattgcccag gccatgagag cacatacttg gagggagaaa |
| | | 661 | aggcttttgt acagcaaaat ggtttgcttt accatctggg tattggcagc tgctctctgc |
| | | 721 | atcccagaaa tcttatacag ccaaatcaag gaggaatccg gcattgctat ctgcaccatg |
| | | 781 | gtttacccta gcgatgagag caccaaactg aagtcagctg tcttgaccct gaaggtcatt |
| | | 841 | ctggggttct tccttccctt cgtggtcatg gcttgctgct ataccatcat cattcacacc |
| | | 901 | ctgatacaag ccaagaagtc ttccaagcac aaagccctaa aagtgaccat cactgtcctg |
| | | 961 | accgtctttg tcttgtctca gtttccctac aactgcattt tgttggtgca gaccattgac |
| | | 1021 | gcctatgcca tgttcatctc caactgtgcc gtttccacca acattgacat ctgcttccag |
| | | 1081 | gtcacccaga ccatcgcctt cttccacagt tgcctgaacc ctgttctcta tgtttttgtg |
| | | 1141 | ggtgagagat tccgccggga tctcgtgaaa accctgaaga acttgggttg catcagccag |
| | | 1201 | gcccagtggg tttcatttac aaggagagag ggaagcttga agctgtcgtc tatgttgctg |
| | | 1261 | gagacaacct caggagcact ctccctctga ggggtcttct ctgaggtgca tggttctttt |
| | | 1321 | ggaagaaatg agaaatacag aaacagtttc cccactgatg ggaccagaga gagtgaaaga |
| | | 1381 | gaaaagaaaa ctcagaaagg gatgaatctg aactatatga ttacttgtag tcagaatttg |
| | | 1441 | ccaaagcaaa tatttcaaaa tcaactgact agtgcaggag gctgttgatt ggctcttgac |
| | | 1501 | tgtgatgccc gcaattctca aaggaggact aaggaccggc actgtggagc accctggctt |
| | | 1561 | tgccactcgc cggagcatca atgccgctgc ctctggagga gcccttggat tttctccatg |
| | | 1621 | cactgtgaac ttctgtggct tcagttctca tgctgcctct tccaaaaggg gacacagaag |
| | | 1681 | cactggctgc tgctacagac cgcaaaagca gaaagtttcg tgaaaatgtc catctttggg |
| | | 1741 | aaattttcta ccctgctctt gagcctgata acccatgcca ggtcttatag attcctgatc |
| | | 1801 | tagaaccttt ccaggcaatc tcagacctaa tttccttctg ttctccttgt tctgttctgg |
| | | 1861 | gccagtgaag gtccttgttc tgattttgaa acgatctgca ggtcttgcca gtgaacccct |
| | | 1921 | ggacaactga ccacacccac aaggcatcca aagtctgttg gcttccaatc catttctgtg |
| | | 1981 | tcctgctgga ggttttaacc tagacaagga ttccgcttat tccttggtat ggtgacagtg |
| | | 2041 | tctctccatg gcctgagcag ggagattata acagctgggt tcgcaggagc cagccttggc |
| | | 2101 | cctgttgtag gcttgttctg ttgagtggca cttgctttgg gtccaccgtc tgtctgctcc |
| | | 2161 | ctagaaaatg ggctggttct tttggccctc ttctttctga ggcccacttt attctgagga |
| | | 2221 | atacagtgag cagatatggg cagcagccag gtagggcaaa ggggtgaagc gcaggccttg |
| | | 2281 | ctggaaggct atttacttcc atgcttctcc ttttcttact ctatagtggc aacattttaa |
| | | 2341 | aagcttttaa cttagagatt aggctgaaaa aaataagtaa tggaattcac ctttgcatct |
| | | 2401 | tttgtgtctt tcttatcatg atttggcaaa atgcatcacc tttgaaaata tttcacatat |
| | | 2461 | tggaaaagtg ctttttaatg tgtatatgaa gcattaatta cttgtcactt tctttaccct |
| | | 2521 | gtctcaatat tttaagtgtg tgcaattaaa gatcaaatag atacatt |
| 6 | NM001256369.1 | 1 | gcttcctttc tcgtgttgtt atcgggtagc tgcctgctca gaacccacaa agcctgcccc |
| | Exemplary nucleic | 61 | tcatcccagg cagagagcaa cccagctctt tccccagaca ctgagagctg gtggtgcctg |
| | acid sequence | 121 | ctgtcccagg gagagttgca tcgccctcca cagagcaggc ttgcatctga ctgacccacc |
| | encoding human | 181 | atgacaccca cagacttcac atctcctcca ggccccgctc cagatcacct tccctcgctg |
| | CCR9 | 241 | gcccaggaat ccatctcctt ccaggacctt agcccaggac taacacaagc cctattccta |
| | | 301 | acatggctga tgactatggc tctgaatcca catcttccat ggaagactac gttaacttca |
| | | 361 | acttcactga cttctactgt gagaaaaaca atgtcaggca gtttgcgagc catttcctcc |
| | | 421 | cacccttgta ctggctcgtg ttcatcgtgg gtgccttggg caacagtctt gttatccttg |
| | | 481 | tctactggta ctgcacaaga gtgaagacca tgaccgacat gttccttttg aatttggcaa |
| | | 541 | ttgctgacct cctctttctt gtcactcttc ccttctgggc cattgctgct gctgaccagt |
| | | 601 | ggaagttcca gaccttcatg tgcaaggtgg tcaacagcat gtacaagatg aacttctaca |
| | | 661 | gctgtgtgtt gctgatcatg tgcatcagcg tggacaggta cattgccatt gcccaggcca |
| | | 721 | tgagagcaca tacttggagg gagaaaaggc ttttgtacag caaaatggtt tgctttacca |
| | | 781 | tctgggtatt ggcagctgct ctctgcatcc cagaaatctt atacagccaa atcaaggagg |
| | | 841 | aatccggcat tgctatctgc accatggttt accctagcga tgagagcacc aaactgaagt |
| | | 901 | cagctgtctt gaccctgaag gtcattctgg ggttcttcct tcccttcgtg gtcatggctt |
| | | 961 | gctgctatac catcatcatt cacaccctga tacaagccaa gaagtcttcc aagcacaaag |
| | | 1021 | ccctaaaagt gaccatcact gtcctgaccg tctttgtctt gtctcagttt ccctacaact |
| | | 1081 | gcattttgtt ggtgcagacc attgacgcct atgccatgtt catctccaac tgtgccgttt |
| | | 1141 | ccaccaacat tgacatctgc ttccaggtca cccagaccat cgccttcttc cacagttgcc |
| | | 1201 | tgaaccctgt tctctatgtt tttgtgggtg agagattccg ccgggatctc gtgaaaaccc |
| | | 1261 | tgaagaactt gggttgcatc agccaggccc agtgggtttc atttacaagg agagagggaa |
| | | 1321 | gcttgaagct gtcgtctatg ttgctggaga caacctcagg agcactctcc ctctgagggg |
| | | 1381 | tcttctctga ggtgcatggt tcttttggaa gaaatgagaa atacagaaac agtttcccca |
| | | 1441 | ctgatgggac cagagagagt gaaagagaaa agaaaactca gaaagggatg aatctgaact |
| | | 1501 | atatgattac ttgtagtcag aatttgccaa agcaaatatt tcaaaatcaa ctgactagtg |
| | | 1561 | caggaggctg ttgattggct cttgactgtg atgcccgcaa ttctcaaagg aggactaagg |
| | | 1621 | accggcactg tggagcaccc tggctttgcc actcgccgga gcatcaatgc cgctgcctct |
| | | 1681 | ggaggagccc ttggattttc tccatgcact gtgaacttct gtggcttcag ttctcatgct |
| | | 1741 | gcctcttcca aaaggggaca cagaagcact ggctgctgct acagaccgca aaagcagaaa |
| | | 1801 | gtttcgtgaa aatgtccatc tttgggaaat tttctaccct gctcttgagc ctgataaccc |
| | | 1861 | atgccaggtc ttatagattc ctgatctaga acctttccag gcaatctcag acctaatttc |
| | | 1921 | cttctgttct ccttgttctg ttctgggcca gtgaaggtcc ttgttctgat tttgaaacga |
| | | 1981 | tctgcaggtc ttgccagtga acccctggac aactgaccac acccacaagg catccaaagt |
| | | 2041 | ctgttggctt ccaatccatt tctgtgtcct gctggaggtt ttaacctaga caaggattcc |
| | | 2101 | gcttattcct tggtatggtg acagtgtctc tccatggcct gagcagggag attataacag |
| | | 2161 | ctgggttcgc aggagccagc cttggccctg ttgtaggctt gttctgttga gtggcacttg |
| | | 2221 | ctttgggtcc accgtctgtc tgctccctag aaaatgggct ggttcttttg gccctcttct |
| | | 2281 | ttctgaggcc cactttattc tgaggaatac agtgagcaga tatgggcagc agccaggtag |
| | | 2341 | ggcaaagggg tgaagcgcag gccttgctgg aaggctattt acttccatgc ttctcctttt |
| | | 2401 | cttactctat agtggcaaca ttttaaaagc ttttaactta gagattaggc tgaaaaaaat |
| | | 2461 | aagtaatgga attcaccttt gcatcttttg tgtctttctt atcatgattt ggcaaaatgc |
| | | 2521 | atcacctttg aaaatatttc acatattgga aaagtgcttt ttaatgtgta tatgaagcat |
| | | 2581 | taattacttg tcactttctt taccctgtct caatatttta agtgtgtgca attaaagatc |
| | | 2641 | aaatagatac att |
| 7 | NP_112477.1 | 1 | mtptdftspi pnmaddygse stssmedyvn fnftdfycek nnvrqfashf 1pplywlvfi |
| | Exemplary amino | 61 | vgalgnslvi lvywyctrvk tmtdmfllnl aiadllflvt lpfwaiaaad qwkfqtfmck |
| | acid sequence for | 121 | vvnsmykmnf yscvllimci svdryiaiaq amrahtwrek rllyskmvcf tiwvlaaalc |
| | human CCR9 | 181 | ipeilysqik eesgiaictm vypsdestkl ksavltlkvi lgfflpfvvm accytiiiht |
| | precursor | 241 | liqakksskh kalkvtitvl tvfvlsqfpy ncillvqtid ayamfisnca vstnidicfq |
| | | 301 | vtqtiaffhs clnpvlyvfv gerfrrdlvk tlknlgcisq aqwvsftrre gslklssmll |
| | | 361 | ettsgalsl |
| 8 | NP_001243298.1 | 1 | maddygsest ssmedyvnfn ftdfyceknn vrqfashflp plywlvfivg algnslvilv |
| | Exemplary amino | 61 | ywyctrvktm tdmfllnlai adllflvtlp fwaiaaadqw kfqtfmckvv nsmykmnfys |
| | acid sequence for | 121 | cvllimcisv dryiaiaqam rahtwrekrl lyskmvcfti wvlaaalcip eilysqikee |
| | human CCR9 | 181 | sgiaictmvy psdestklks avltlkvilg fflpfvvmac cytiiihtli qakksskhka |
| | precursor | 241 | lkvtitvltv fvlsqfpync illvqtiday amfisncavs tnidicfqvt qtiaffhscl |
| | | 301 | npvlyvfvge rfrrdlvktl knlgcisqaq wvsftrregs lklssmllet tsgalsl |
| 9 | NM_000885.4 | 1 | ataacgtctt tgtcactaaa atgttcccca ggggccttcg gcgagtcttt ttgtttggtt |
| | Exemplary nucleic | 61 | ttttgttttt aatctgtggc tcttgataat ttatctagtg gttgcctaca cctgaaaaac |
| | acid sequence | 121 | aagacacagt gtttaactat caacgaaaga actggacggc tccccgccgc agtcccactc |
| | encoding human | 181 | cccgagtttg tggctggcat ttgggccacg ccgggctggg cggtcacagc gaggggcgcg |
| | α4 | 241 | cagtttgggg tcacacagct ccgcttctag gccccaacca ccgttaaaag gggaagcccg |
| | | 301 | tgccccatca ggtccgctct tgctgagccc agagccatcc cgcgctctgc gggctgggag |
| | | 361 | gcccgggcca ggacgcgagt cctgcgcagc cgaggttccc cagcgccccc tgcagccgcg |
| | | 421 | cgtaggcaga gacggagccc ggccctgcgc ctccgcacca cgcccgggac cccacccagc |
| | | 481 | ggcccgtacc cggagaagca gcgcgagcac ccgaagctcc cggctggcgg cagaaaccgg |
| | | 541 | gagtggggcc gggcgagtgc gcggcatccc aggccggccc gaacgctccg cccgcggtgg |
| | | 601 | gccgacttcc cctcctcttc cctctctcct tcctttagcc cgctggcgcc ggacacgctg |
| | | 661 | cgcctcatct cttggggcgt tcttccccgt tggccaaccg tcgcatcccg tgcaactttg |
| | | 721 | gggtagtggc cgtttagtgt tgaatgttcc ccaccgagag cgcatggctt gggaagcgag |
| | | 781 | gcgcgaaccc ggcccccgaa gggccgccgt ccgggagacg gtgatgctgt tgctgtgcct |
| | | 841 | gggggtcccg accggccgcc cctacaacgt ggacactgag agcgcgctgc tttaccaggg |
| | | 901 | cccccacaac acgctgttcg gctactcggt cgtgctgcac agccacgggg cgaaccgatg |
| | | 961 | gctcctagtg ggtgcgccca ctgccaactg gctcgccaac gcttcagtga tcaatcccgg |
| | | 1021 | ggcgatttac agatgcagga tcggaaagaa tcccggccag acgtgcgaac agctccagct |
| | | 1081 | gggtagccct aatggagaac cttgtggaaa gacttgtttg gaagagagag acaatcagtg |
| | | 1141 | gttgggggtc acactttcca gacagccagg agaaaatgga tccatcgtga cttgtgggca |
| | | 1201 | tagatggaaa aatatatttt acataaagaa tgaaaataag ctccccactg gtggttgcta |
| | | 1261 | tggagtgccc cctgatttac gaacagaact gagtaaaaga atagctccgt gttatcaaga |
| | | 1321 | ttatgtgaaa aaatttggag aaaattttgc atcatgtcaa gctggaatat ccagttttta |
| | | 1381 | cacaaaggat ttaattgtga tgggggcccc aggatcatct tactggactg gctctctttt |
| | | 1441 | tgtctacaat ataactacaa ataaatacaa ggctttttta gacaaacaaa atcaagtaaa |
| | | 1501 | atttggaagt tatttaggat attcagtcgg agctggtcat tttcggagcc agcatactac |
| | | 1561 | cgaagtagtc ggaggagctc ctcaacatga gcagattggt aaggcatata tattcagcat |
| | | 1621 | tgatgaaaaa gaactaaata tcttacatga aatgaaaggt aaaaagcttg gatcgtactt |
| | | 1681 | tggagcttct gtctgtgctg tggacctcaa tgcagatggc ttctcagatc tgctcgtggg |
| | | 1741 | agcacccatg cagagcacca tcagagagga aggaagagtg tttgtgtaca tcaactctgg |
| | | 1801 | ctcgggagca gtaatgaatg caatggaaac aaacctcgtt ggaagtgaca aatatgctgc |
| | | 1861 | aagatttggg gaatctatag ttaatcttgg cgacattgac aatgatggct ttgaagatgt |
| | | 1921 | tgctatcgga gctccacaag aagatgactt gcaaggtgct atttatattt acaatggccg |
| | | 1981 | tgcagatggg atctcgtcaa ccttctcaca gagaattgaa ggacttcaga tcagcaaatc |
| | | 2041 | gttaagtatg tttggacagt ctatatcagg acaaattgat gcagataata atggctatgt |
| | | 2101 | agatgtagca gttggtgctt ttcggtctga ttctgctgtc ttgctaagga caagacctgt |
| | | 2161 | agtaattgtt gacgcttctt taagccaccc tgagtcagta aatagaacga aatttgactg |
| | | 2221 | tgttgaaaat ggatggcctt ctgtgtgcat agatctaaca ctttgtttct catataaggg |
| | | 2281 | caaggaagtt ccaggttaca ttgttttgtt ttataacatg agtttggatg tgaacagaaa |
| | | 2341 | ggcagagtct ccaccaagat tctatttctc ttctaatgga acttctgacg tgattacagg |
| | | 2401 | aagcatacag gtgtccagca gagaagctaa ctgtagaaca catcaagcat ttatgcggaa |
| | | 2461 | agatgtgcgg gacatcctca ccccaattca gattgaagct gcttaccacc ttggtcctca |
| | | 2521 | tgtcatcagt aaacgaagta cagaggaatt cccaccactt cagccaattc ttcagcagaa |
| | | 2581 | gaaagaaaaa gacataatga aaaaaacaat aaactttgca aggttttgtg cccatgaaaa |
| | | 2641 | ttgttctgct gatttacagg tttctgcaaa gattgggttt ttgaagcccc atgaaaataa |
| | | 2701 | aacatatctt gctgttggga gtatgaagac attgatgttg aatgtgtcct tgtttaatgc |
| | | 2761 | tggagatgat gcatatgaaa cgactctaca tgtcaaacta cccgtgggtc tttatttcat |
| | | 2821 | taagatttta gagctggaag agaagcaaat aaactgtgaa gtcacagata actctggcgt |
| | | 2881 | ggtacaactt gactgcagta ttggctatat atatgtagat catctctcaa ggatagatat |
| | | 2941 | tagctttctc ctggatgtga gctcactcag cagagcggaa gaggacctca gtatcacagt |
| | | 3001 | gcatgctacc tgtgaaaatg aagaggaaat ggacaatcta aagcacagca gagtgactgt |
| | | 3061 | agcaatacct ttaaaatatg aggttaagct gactgttcat gggtttgtaa acccaacttc |
| | | 3121 | atttgtgtat ggatcaaatg atgaaaatga gcctgaaacg tgcatggtgg agaaaatgaa |
| | | 3181 | cttaactttc catgttatca acactggcaa tagtatggct cccaatgtta gtgtggaaat |
| | | 3241 | aatggtacca aattctttta gcccccaaac tgataagctg ttcaacattt tggatgtcca |
| | | 3301 | gactactact ggagaatgcc actttgaaaa ttatcaaaga gtgtgtgcat tagagcagca |
| | | 3361 | aaagagtgca atgcagacct tgaaaggcat agtccggttc ttgtccaaga ctgataagag |
| | | 3421 | gctattgtac tgcataaaag ctgatccaca ttgtttaaat ttcttgtgta attttgggaa |
| | | 3481 | aatggaaagt ggaaaagaag ccagtgttca tatccaactg gaaggccggc catccatttt |
| | | 3541 | agaaatggat gagacttcag cactcaagtt tgaaataaga gcaacaggtt ttccagagcc |
| | | 3601 | aaatccaaga gtaattgaac taaacaagga tgagaatgtt gcgcatgttc tactggaagg |
| | | 3661 | actacatcat caaagaccca aacgttattt caccatagtg attatttcaa gtagcttgct |
| | | 3721 | acttggactt attgtacttc tgttgatctc atatgttatg tggaaggctg gcttctttaa |
| | | 3781 | aagacaatac aaatctatcc tacaagaaga aaacagaaga gacagttgga gttatatcaa |
| | | 3841 | cagtaaaagc aatgatgatt aaggacttct ttcaaattga gagaatggaa aacagactca |
| | | 3901 | ggttgtagta aagaaattta aaagacactg tttacaagaa aaaatgaatt ttgtttggac |
| | | 3961 | ttcttttact catgatcttg tgacatatta tgtcttcatg caaggggaaa atctcagcaa |
| | | 4021 | tgattactct ttgagataga agaactgcaa aggtaataat acagccaaag ataatctctc |
| | | 4081 | agcttttaaa tgggtagaga aacactaaag cattcaattt attcaagaaa agtaagccct |
| | | 4141 | tgaagatatc ttgaaatgaa agtataactg agttaaatta tactggagaa gtcttagact |
| | | 4201 | tgaaatacta cttaccatat gtgcttgcct cagtaaaatg aaccccactg ggtgggcaga |
| | | 4261 | ggttcatttc aaatacatct ttgatacttg ttcaaaatat gttctttaaa aatataattt |
| | | 4321 | tttagagagc tgttcccaaa ttttctaacg agtggaccat tatcacttta aagcccttta |
| | | 4381 | tttataatac atttcctacg ggctgtgttc caacaaccat tttttttcag cagactatga |
| | | 4441 | atattatagt attataggcc aaactggcaa acttcagact gaacatgtac actggtttga |
| | | 4501 | gcttagtgaa attacttctg gataattatt tttttataat tatggatttc accatctttc |
| | | 4561 | tttctgtata tatacatgtg tttttatgta ggtatatatt taccattctt cctatctatt |
| | | 4621 | cttcctataa cacaccttta tcaagcatac ccaggagtaa tcttcaaatc ttttgttata |
| | | 4681 | ttctgaaaca aaagattgtg agtgttgcac tttacctgat acacgctgat ttagaaaata |
| | | 4741 | cagaaaccat acctcactaa taactttaaa atcaaagctg tgcaaagact agggggccta |
| | | 4801 | tacttcatat gtattatgta ctatgtaaaa tattgactat cacacaacta tttccttgga |
| | | 4861 | tgtaattctt tgttaccctt tacaagtata agtgttacct tacatggaaa cgaagaaaca |
| | | 4921 | aaattcataa atttaaattc ataaatttag ctgaaagata ctgattcaat ttgtatacag |
| | | 4981 | tgaatataaa tgagacgaca gcaaaatttt catgaaatgt aaaatatttt tatagtttgt |
| | | 5041 | tcatactata tgaggttcta ttttaaatga ctttctggat tttaaaaaat ttctttaaat |
| | | 5101 | acaatcattt ttgtaatatt tattttatgc ttatgatcta gataattgca gaatatcatt |
| | | 5161 | ttatctgact ctgccttcat aagagagctg tggccgaatt ttgaacatct gttataggga |
| | | 5221 | gtgatcaaat tagaaggcaa tgtggaaaaa caattctggg aaagatttct ttatatgaag |
| | | 5281 | tccctgccac tagccagcca tcctaattga tgaaagttat ctgttcacag gcctgcagtg |
| | | 5341 | atggtgagga atgttctgag atttgcgaag gcatttgagt agtgaaatgt aagcacaaaa |
| | | 5401 | cctcctgaac ccagagtgtg tatacacagg aataaacttt atgacattta tgtattttta |
| | | 5461 | aaaaactttg tatcgttata aaaaggctag tcattctttc aggagaacat ctaggatcat |
| | | 5521 | agatgaaaaa tcaagccccg atttagaact gtcttctcca ggatggtctc taaggaaatt |
| | | 5581 | tacatttggt tctttcctac tcagaactac tcagaaacaa ctatatattt caggttatct |
| | | 5641 | gagcacagtg aaagcagagt actatggttg tccaacacag gcctctcaga tacaagggga |
| | | 5701 | acacaattac atattgggct agattttgcc cagttcaaaa tagtatttgt tatcaactta |
| | | 5761 | ctttgttact tgtatcatga attttaaaac cctaccactt taagaagaca gggatgggtt |
| | | 5821 | attctttttt ggcaggtagg ctatataact atgtgatttt gaaatttaac tgctctggat |
| | | 5881 | tagggagcag tgaatcaagg cagacttatg aaatctgtat tatatttgta acagaatata |
| | | 5941 | ggaaatttaa cataattgat gagctcaaat cctgaaaaat gaaagaatcc aaattatttc |
| | | 6001 | agaattatct aggttaaata ttgatgtatt atgatggttg caaagttttt ttgtgtgtcc |
| | | 6061 | aataaacaca ttgtaaaaaa aa |
| 10 | NM_000889.2 | 1 | aaatcttccc caccctgggg agtgtcactt cctcctctgc cgtctcccag atcagtacac |
| | Exemplary nucleic | 61 | aaaggctgct gctgccgcca gaggaaggac tgctctgcac gcacctatgt ggaaactaaa |
| | acid sequence | 121 | gcccagagag aaagtctgac ttgccccaca gccagtgagt gactgcagca gcaccagaat |
| | encoding human | 181 | ctggtctgtt tcctgtttgg ctcttctacc actacggctt gggatctcgg gcatggtggc |
| | β7 | 241 | tttgccaatg gtccttgttt tgctgctggt cctgagcaga ggtgagagtg aattggacgc |
| | | 301 | caagatccca tccacagggg atgccacaga atggcggaat cctcacctgt ccatgctggg |
| | | 361 | gtcctgccag ccagccccct cctgccagaa gtgcatcctc tcacacccca gctgtgcatg |
| | | 421 | gtgcaagcaa ctgaacttca ccgcgtcggg agaggcggag gcgcggcgct gcgcccgacg |
| | | 481 | agaggagctg ctggctcgag gctgcccgct ggaggagctg gaggagcccc gcggccagca |
| | | 541 | ggaggtgctg caggaccagc cgctcagcca gggcgcccgc ggagagggtg ccacccagct |
| | | 601 | ggcgccgcag cgggtccggg tcacgctgcg gcctggggag ccccagcagc tccaggtccg |
| | | 661 | cttccttcgt gctgagggat acccggtgga cctgtactac cttatggacc tgagctactc |
| | | 721 | catgaaggac gacctggaac gcgtgcgcca gctcgggcac gctctgctgg tccggctgca |
| | | 781 | ggaagtcacc cattctgtgc gcattggttt tggttccttt gtggacaaaa cggtgctgcc |
| | | 841 | ctttgtgagc acagtaccct ccaaactgcg ccacccctgc cccacccggc tggagcgctg |
| | | 901 | ccagtcacca ttcagctttc accatgtgct gtccctgacg ggggacgcac aagccttcga |
| | | 961 | gcgggaggtg gggcgccaga gtgtgtccgg caatctggac tcgcctgaag gtggcttcga |
| | | 1021 | tgccattctg caggctgcac tctgccagga gcagattggc tggagaaatg tgtcccggct |
| | | 1081 | gctggtgttc acttcagacg acacattcca tacagctggg gacgggaagt tgggcggcat |
| | | 1141 | tttcatgccc agtgatgggc actgccactt ggacagcaat ggcctctaca gtcgcagcac |
| | | 1201 | agagtttgac tacccttctg tgggtcaggt agcccaggcc ctctctgcag caaatatcca |
| | | 1261 | gcccatcttt gctgtcacca gtgccgcact gcctgtctac caggagctga gtaaactgat |
| | | 1321 | tcctaagtct gcagttgggg agctgagtga ggactccagc aacgtggtac agctcatcat |
| | | 1381 | ggatgcttat aatagcctgt cttccaccgt gacccttgaa cactcttcac tccctcctgg |
| | | 1441 | ggtccacatt tcttacgaat cccagtgtga gggtcctgag aagagggagg gtaaggctga |
| | | 1501 | ggatcgagga cagtgcaacc acgtccgaat caaccagacg gtgactttct gggtttctct |
| | | 1561 | ccaagccacc cactgcctcc cagagcccca tctcctgagg ctccgggccc ttggcttctc |
| | | 1621 | agaggagctg attgtggagt tgcacacgct gtgtgactgt aattgcagtg acacccagcc |
| | | 1681 | ccaggctccc cactgcagtg atggccaggg acacctacaa tgtggtgtat gcagctgtgc |
| | | 1741 | ccctggccgc ctaggtcggc tctgtgagtg ctctgtggca gagctgtcct ccccagacct |
| | | 1801 | ggaatctggg tgccgggctc ccaatggcac agggcccctg tgcagtggaa agggtcactg |
| | | 1861 | tcaatgtgga cgctgcagct gcagtggaca gagctctggg catctgtgcg agtgtgacga |
| | | 1921 | tgccagctgt gagcgacatg agggcatcct ctgcggaggc tttggtcgct gccaatgtgg |
| | | 1981 | agtatgtcac tgtcatgcca accgcacggg cagagcatgc gaatgcagtg gggacatgga |
| | | 2041 | cagttgcatc agtcccgagg gagggctctg cagtgggcat ggacgctgca aatgcaaccg |
| | | 2101 | ctgccagtgc ttggacggct actatggtgc tctatgcgac caatgcccag gctgcaagac |
| | | 2161 | accatgcgag agacaccggg actgtgcaga gtgtggggcc ttcaggactg gcccactggc |
| | | 2221 | caccaactgc agtacagctt gtgcccatac caatgtgacc ctggccttgg cccctatctt |
| | | 2281 | ggatgatggc tggtgcaaag agcggaccct ggacaaccag ctgttcttct tcttggtgga |
| | | 2341 | ggatgacgcc agaggcacgg tcgtgctcag agtgagaccc caagaaaagg gagcagacca |
| | | 2401 | cacgcaggcc attgtgctgg gctgcgtagg gggcatcgtg gcagtggggc tggggctggt |
| | | 2461 | cctggcttac cggctctcgg tggaaatcta tgaccgccgg gaatacagtc gctttgagaa |
| | | 2521 | ggagcagcaa caactcaact ggaagcagga cagtaatcct ctctacaaaa gtgccatcac |
| | | 2581 | gaccaccatc aatcctcgct ttcaagaggc agacagtccc actctctgaa ggagggaggg |
| | | 2641 | acacttaccc aaggctcttc tccttggagg acagtgggaa ctggagggtg agaggaaggg |
| | | 2701 | tgggtctgta agaccttggt aggggactaa ttcactggcg aggtgcggcc accaccctac |
| | | 2761 | ttcattttca gagtgacacc caagagggct gcttcccatg cctgcaacct tgcatccatc |
| | | 2821 | tgggctaccc cacccaagta tacaataaag tcttacctca gaccacaaaa aaaaaaaa |
| 11 | NP_000876.3 | 1 | mawearrepg prraavretv mlllclgvpt grpynvdtes allyqgphnt lfgysvvlhs |
| | Exemplary amino | 61 | hganrwllvg aptanwlana svinpgaiyr crigknpgqt ceqlqlgspn gepcgktcle |
| | acid sequence for | 121 | erdnqwlgvt lsrqpgengs ivtcghrwkn ifyiknenkl ptggcygvpp dlrtelskri |
| | human α4 | 181 | apcyqdyvkk fgenfascqa gissfytkdl ivmgapgssy wtgslfvyni ttnkykafld |
| | precursor | 241 | kqnqvkfgsy lgysvgaghf rsqhttevvg gapqheqigk ayifsideke lnilhemkgk |
| | | 301 | klgsyfgasv cavdlnadgf sdllvgapmq stireegrvf vyinsgsgav mnametnlvg |
| | | 361 | sdkyaarfge sivnlgdidn dgfedvaiga pqeddlqgai yiyngradgi sstfsqrieg |
| | | 421 | lqiskslsmf gqsisgqida dnngyvdvav gafrsdsavl lrtrpvvivd aslshpesvn |
| | | 481 | rtkfdcveng wpsvcidltl cfsykgkevp gyivlfynms ldvnrkaesp prfyfssngt |
| | | 541 | sdvitgsiqv ssreancrth qafmrkdvrd iltpiqieaa yhlgphvisk rsteefpplq |
| | | 601 | pilqqkkekd imkktinfar fcahencsad lqvsakigfl kphenktyla vgsmktlmln |
| | | 661 | vslfnagdda yettlhvklp vglyfikile leekqincev tdnsgvvqld csigyiyvdh |
| | | 721 | lsridisfll dvsslsraee dlsitvhatc eneeemdnlk hsrvtvaipl kyevkltvhg |
| | | 781 | fvnptsfvyg sndenepetc mvekmnltfh vintgnsmap nvsveimvpn sfspqtdklf |
| | | 841 | nildvqtttg echfenyqrv caleqqksam qtlkgivrfl sktdkrllyc ikadphclnf |
| | | 901 | lcnfgkmesg keasvhiqle grpsilemde tsalkfeira tgfpepnprv ielnkdenva |
| | | 961 | hvlleglhhq rpkryftivi issslllgli vlllisyvmw kagffkrqyk silqeenrrd |
| | | 1021 | swsyinsksn dd |
| 12 | NP_000880.1 | 1 | mvalpmvlvl llvlsrgese ldakmpstgd atewrnphls mlgscqpaps cqkcilshps |
| | Exemplary amino | 61 | cawckqlnft asgeaearrc arreellarg cpleeleepr gqqevlqdqp lsqgargega |
| | acid sequence for | 121 | tqlapqrvrv tlrpgepqql qvrflraegy pvdlyylmdl sysmkddler vrqlghallv |
| | human β7 | 181 | rlqevthsvr igfgsfvdkt vlpfvstvps klrhpcptrl ercqspfsfh hvlsltgdaq |
| | precursor | 241 | aferevgrqs vsgnldspeg gfdailqaal cqeqigwrnv srllvftsdd tfhtagdgkl |
| | | 301 | ggifmpsdgh chldsnglys rstefdypsv gqvaqalsaa niqpifavts aalpvyqels |
| | | 361 | klipksavge lsedssnvvq limdaynsls stvtlehssl ppgvhisyes qcegpekreg |
| | | 421 | kaedrgqcnh vrinqtvtfw vslqathclp ephllrlral gfseelivel htlcdcncsd |
| | | 481 | tqpqaphcsd gqghlqcgvc scapgrlgrl cecsvaelss pdlesgcrap ngtgplcsgk |
| | | 541 | ghcqcgrcsc sgqssghlce cddascerhe gilcggfgrc qcgvchchan rtgracecsg |
| | | 601 | dmdscispeg glcsghgrck cnrcqcldgy ygalcdqcpg cktpcerhrd caecgafrtg |
| | | 661 | platncstac ahtnvtlala pilddgwcke rtldnqlfff lveddargtv vlrvrpqekg |
| | | 721 | adhtqaivlg cvggivavgl glvlayrlsv eiydrreysr fekeqqqlnw kqdsnplyks |
| | | 781 | aitttinprf qeadsptl |
| 13 | NM_016602.2 | 1 | agagatgggg acggaggcca cagagcaggt ttcctggggc cattactctg gggatgaaga |
| | Exemplary nucleic | 61 | ggacgcatac tcggctgagc cactgccgga gctttgctac aaggccgatg tccaggcctt |
| | acid sequence | 121 | cagccgggcc ttccaaccca gtgtctccct gaccgtggct gcgctgggtc tggccggcaa |
| | encoding human | 181 | tggcctggtc ctggccaccc acctggcagc ccgacgcgca gcgcgctcgc ccacctctgc |
| | CCR10 | 241 | ccacctgctc cagctggccc tggccgacct cttgctggcc ctgactctgc ccttcgcggc |
| | | 301 | agcaggggct cttcagggct ggagtctggg aagtgccacc tgccgcacca tctctggcct |
| | | 361 | ctactcggcc tccttccacg ccggcttcct cttcctggcc tgtatcagcg ccgaccgcta |
| | | 421 | cgtggccatc gcgcgagcgc tcccagccgg gccgcggccc tccactcccg gccgcgcaca |
| | | 481 | cttggtctcc gtcatcgtgt ggctgctgtc actgctcctg gcgctgcctg cgctgctctt |
| | | 541 | cagccaggat gggcagcggg aaggccaacg acgctgtcgc ctcatcttcc ccgagggcct |
| | | 601 | cacgcagacg gtgaaggggg cgagcgccgt ggcgcaggtg gccctgggct tcgcgctgcc |
| | | 661 | gctgggcgtc atggtagcct gctacgcgct tctgggccgc acgctgctgg ccgccagggg |
| | | 721 | gcccgagcgc cggcgtgcgc tgcgcgtcgt ggtggctctg gtggcggcct tcgtggtgct |
| | | 781 | gcagctgccc tacagcctcg ccctgctgct ggatactgcc gatctactgg ctgcgcgcga |
| | | 841 | gcggagctgc cctgccagca aacgcaagga tgtcgcactg ctggtgacca gcggcttggc |
| | | 901 | cctcgcccgc tgtggcctca atcccgttct ctacgccttc ctgggcctgc gcttccgcca |
| | | 961 | ggacctgcgg aggctgctac ggggtgggag ctgcccctca gggcctcaac cccgccgcgg |
| | | 1021 | ctgcccccgc cggccccgcc tttcttcctg ctcagctccc acggagaccc acagtctctc |
| | | 1081 | ctgggacaac tagggctgcg aatctagagg agggggcagg ctgagggtcg tgggaaaggg |
| | | 1141 | gagtaggtgg gggaacactg agaaagaggc agggacctaa agggactacc tctgtgcctt |
| | | 1201 | gccacattaa attgataaca tggaaatgag atgcaaccca acaa |
| 14 | AF215981.1 | 1 | agagatgggg acggaggcca cagagcaggt ttcctggggc cattactctg gggatgaaga |
| | Exemplary nucleic | 61 | ggacgcatac tcggctgagc cactgccgga gctttgctac aaggccgatg tccaggcctt |
| | acid sequence | 121 | cagccgggcc ttccaaccca gtgtctccct gaccgtggct gcgctgggtc tggccggcaa |
| | encoding human | 181 | tggcctggtc ctggccaccc acctggcagc ccgacgcgca gcgcgctcgc ccacctctgc |
| | CCR10 | 241 | ccacctgctc cagctggccc tggccgacct cttgctggcc ctgactctgc ccttcgcggc |
| | | 301 | agcaggggct cttcagggct ggagtctggg aagtgccacc tgccgcacca tctctggcct |
| | | 361 | ctactcggcc tccttccacg ccggcttcct cttcctggcc tgtatcagcg ccgaccgcta |
| | | 421 | cgtggccatc gcgcgagcgc tcccagccgg gccgcggccc tccactcccg gccgcgcaca |
| | | 481 | cttggtctcc gtcatcgtgt ggctgctgtc actgctcctg gcgctgcctg cgctgctctt |
| | | 541 | cagccaggat gggcagcggg aaggccaacg acgctgtcgc ctcatcttcc ccgagggcct |
| | | 601 | cacgcagacg gtgaaggggg cgagcgccgt ggcgcaggtg gccctgggct tcgcgctgcc |
| | | 661 | gctgggcgtc atggtagcct gctacgcgct tctgggccgc acgctgctgg ccgccagggg |
| | | 721 | gcccgagcgc cggcgtgcgc tgcgcgtcgt ggtggctctg gtggcggcct tcgtggtgct |
| | | 781 | gcagctgccc tacagcctcg ccctgctgct ggatactgcc gatctactgg ctgcgcgcga |
| | | 841 | gcggagctgc cctgccagca aacgcaagga tgtcgcactg ctggtgacca gcggcttggc |
| | | 901 | cctcgcccgc tgtggcctca atcccgttct ctacgccttc ctgggcctgc gcttccgcca |
| | | 961 | ggacctgcgg aggctgctac ggggtgggag ctcgccctca gggcctcaac cccgccgcgg |
| | | 1021 | ctgcccccgc cggccccgcc tttcttcctg ctcagctccc acggagaccc acagtctctc |
| | | 1081 | ctgggacaac tagggctgcg aatctagagg agggggcagg ctgagggtcg tgggaaaggg |
| | | 1141 | gagtaggtgg gggaacactg agaaagaggc agggacctaa agggactacc tctgtgcctt |
| | | 1201 | gccacattaa attgataaca tggaaatgaa aaaaaaaaaa aaaa |
| 15 | NP_057686.2 | 1 | mgteateqvs wghysgdeed aysaeplpel cykadvqafs rafqpsyslt vaalglagng |
| | Exemplary amino | 61 | lvlathlaar raarsptsah llqlaladll laltlpfaaa galqgwslgs atcrtisgly |
| | acid sequence for | 121 | sasfhagflf lacisadryv aiaralpagp rpstpgrahl vsvivwllsl llalpallfs |
| | human CCR10 | 181 | qdgqregqrr crlifpeglt qtvkgasava qvalgfalpl gvmvacyall grtllaargp |
| | precursor | 241 | errralrvvv alvaafvvlq lpyslallld tadllaarer scpaskrkdv allvtsglal |
| | | 301 | arcglnpvly aflglrfrqd lrrllrggsc psgpqprrgc prrprlsscs aptethslsw |
| | | 361 | dn |
| 16 | P46092.3 | 1 | mgteateqvs wghysgdeed aysaeplpel cykadvqafs rafqpsyslt vaalglagng |
| | Exemplary amino | 61 | lvlathlaar raarsptsah llqlaladll laltlpfaaa galqgwslgs atcrtisgly |
| | acid sequence for | 121 | sasfhagflf lacisadryv aiaralpagp rpstpgrahl vsvivwllsl llalpallfs |
| | human CCR10 | 181 | qdgqregqrr crlifpeglt qtvkgasava qvalgfalpl gvmvacyall grtllaargp |
| | precursor | 241 | errralrvvv alvaafvvlq lpyslallld tadllaarer scpaskrkdv allvtsglal |
| | | 301 | arcglnpvly aflglrfrqd lrrllrggsc psgpqprrgc prrprlsscs aptethslsw |
| | | 361 | dn |
| 17 | NM_005201.3 | 1 | tttgtagtgg gaggatacct ccagagaggc tgctgctcat tgagctgcac tcacatgagg |
| | Exemplary nucleic | 61 | atacagactt tgtgaagaag gaattggcaa cactgaaacc tccagaacaa aggctgtcac |
| | acid sequence | 121 | taaggtcccg ctgccttgat ggattataca cttgacctca gtgtgacaac agtgaccgac |
| | encoding human | 181 | tactactacc ctgatatctt ctcaagcccc tgtgatgcgg aacttattca gacaaatggc |
| | CCR8 | 241 | aagttgctcc ttgctgtctt ttattgcctc ctgtttgtat tcagtcttct gggaaacagc |
| | | 301 | ctggtcatcc tggtccttgt ggtctgcaag aagctgagga gcatcacaga tgtatacctc |
| | | 361 | ttgaacctgg ccctgtctga cctgcttttt gtcttctcct tcccctttca gacctactat |
| | | 421 | ctgctggacc agtgggtgtt tgggactgta atgtgcaaag tggtgtctgg cttttattac |
| | | 481 | attggcttct acagcagcat gtttttcatc accctcatga gtgtggacag gtacctggct |
| | | 541 | gttgtccatg ccgtgtatgc cctaaaggtg aggacgatca ggatgggcac aacgctgtgc |
| | | 601 | ctggcagtat ggctaaccgc cattatggct accatcccat tgctagtgtt ttaccaagtg |
| | | 661 | gcctctgaag atggtgttct acagtgttat tcattttaca atcaacagac tttgaagtgg |
| | | 721 | aagatcttca ccaacttcaa aatgaacatt ttaggcttgt tgatcccatt caccatcttt |
| | | 781 | atgttctgct acattaaaat cctgcaccag ctgaagaggt gtcaaaacca caacaagacc |
| | | 841 | aaggccatca ggttggtgct cattgtggtc attgcatctt tacttttctg ggtcccattc |
| | | 901 | aacgtggttc ttttcctcac ttccttgcac agtatgcaca tcttggatgg atgtagcata |
| | | 961 | agccaacagc tgacttatgc cacccatgtc acagaaatca tttcctttac tcactgctgt |
| | | 1021 | gtgaaccctg ttatctatgc ttttgttggg gagaagttca agaaacacct ctcagaaata |
| | | 1081 | tttcagaaaa gttgcagcca aatcttcaac tacctaggaa gacaaatgcc tagggagagc |
| | | 1141 | tgtgaaaagt catcatcctg ccagcagcac tcctcccgtt cctccagcgt agactacatt |
| | | 1201 | ttgtgaggat caatgaagac taaatataaa aaacattttc ttgaatggca tgctagtagc |
| | | 1261 | agtgagcaaa ggtgtgggtg tgaaaggttt ccaaaaaaag ttcagcatga aggatgccat |
| | | 1321 | atatgttgtt gccaacactt ggaacacaat gactaaagac atagttgtgc atgcctggca |
| | | 1381 | caacatcaag cctgtgattg tgtttattga tgatgttgaa caagtggtaa ctttaaagga |
| | | 1441 | ttctgtatgc caagtgaaaa aaaaagatgt ctgacctcct tacatat |
| 18 | BC107159.1 | 1 | ctttgtgaag aaggaattgg caacactgaa acctccagaa caaaggctgt cactaaggtc |
| | Exemplary nucleic | 61 | ccgctgcctt gatggattat acacttgacc tcagtgtgac aacagtgacc gactactact |
| | acid sequence | 121 | accctgatat cttctcaagc ccctgtgatg cggaacttat tcagacaaat ggcaagttgc |
| | encoding human | 181 | tccttgctgt cttttattgc ctcctgtttg tattcagtct tctgggaaac agcctggtca |
| | CCR8 | 241 | tcctggtcct tgtggtctgc aagaagctga ggagcatcac agatgtatac ctcttgaacc |
| | | 301 | tggccctgtc tgacctgctt tttgtcttct ccttcccctt tcagacctac tatctgctgg |
| | | 361 | accagtgggt gtttgggact gtaatgtgca aagtggtgtc tggcttttat tacattggct |
| | | 421 | tctacagcag catgtttttc atcaccctca tgagtgtgga caggtacctg gctgttgtcc |
| | | 481 | atgccgtgta tgccctaaag gtgaggacga tcaggatggg cacaacgctg tgcctggcag |
| | | 541 | tatggctaac cgccattatg gctaccatcc cattgctagt gttttaccaa gtggcctctg |
| | | 601 | aagatggtgt tctacagtgt tattcatttt acaatcaaca gactttgaag tggaagatct |
| | | 661 | tcaccaactt caaaatgaac attttaggct tgttgatccc attcaccatc tttatgttct |
| | | 721 | gctacattaa aatcctgcac cagctgaaga ggtgtcaaaa ccacaacaag accaaggcca |
| | | 781 | tcaggttggt gctcattgtg gtcattgcat ctttactttt ctgggtccca ttcaacgtgg |
| | | 841 | ttcttttcct cacttccttg cacagtatgc acatcttgga tggatgtagc ataagccaac |
| | | 901 | agctgactta tgccacccat gtcacagaaa tcatttcctt tactcactgc tgtgtgaacc |
| | | 961 | ctgttatcta tgcttttgtt ggggagaagt tcaagaaaca cctctcagaa atatttcaga |
| | | 1021 | aaagttgcag ccaaatcttc aactacctag gaagacaaat gcctagggag agctgtgaaa |
| | | 1081 | agtcatcatc ctgccagcag cactcctccc gttcctccag cgtagactac attttgtgag |
| | | 1141 | gatcaatgaa gactaaatat aaaaaacatt ttcttgaatg gcatgctagt agcagtgagc |
| | | 1201 | aaaggtgtgg gtgtgaaagg tttccaaaaa aagttcagca tgaaggatgc cgtgtgtgtt |
| | | 1261 | gttgccaaca cttggaacac gatgactggg gacgtggttg tgcatgcctg gcacaacatc |
| | | 1321 | aagcctgtga ttgtgtttat tgatgatgtt gaacaagtgg tggctttgga ggattctgta |
| | | 1381 | tgccaagtga aaggggagat gtctgacctc cttcatatag |
| 19 | NP_005192.1 | 1 | mdytldlsvt tvtdyyypdi fsspcdaeli qtngkillav fycllfvfsl lgnslvilvl |
| | Exemplary amino | 61 | vvckklrsit dvyllnlals dllfvfsfpf qtyylldqwv fgtvmckvvs gfyyigfyss |
| | acid sequence for | 121 | mffitlmsvd rylavvhavy alkvrtirmg ttlclavwlt aimatipllv fyqvasedgv |
| | human CCR8 | 181 | lqcysfynqq tlkwkiftnf kmnilgllip ftifmfcyik ilhqlkrcqn hnktkairlv |
| | precursor | 241 | livviasllf wvpfnvvlfl tslhsmhild gcsisqqlty athvteiisf thccvnpviy |
| | | 301 | afvgekfkkh lseifqkscs qifnylgrqm presceksss cqqhssrsss vdyil |
| 20 | AAI07160A | 1 | mdytldlsvt tvtdyyypdi fsspcdaeli qtngklllav fycllfvfsl lgnslvilvl |
| | Exemplary amino | 61 | vvckklrsit dvyllnlals dllfvfsfpf qtyylldqwv fgtvmckvvs gfyyigfyss |
| | acid sequence for | 121 | mffitlmsvd rylavvhavy alkvrtirmg ttlclavwlt aimatipllv fyqvasedgv |
| | human CCR8 | 181 | lqcysfynqq tlkwkiftnf kmnilgllip ftifmfcyik ilhqlkrcqn hnktkairlv |
| | precursor | 241 | livviasllf wvpfnvvlfl tslhsmhild gcsisqqlty athvteiisf thccvnpviy |
| | | 301 | afvgekfkkh lseifqkscs qifnylgrqm presceksss cqqhssrsss vdyil |
| 21 | NM_005508.4 | 1 | gctcacagga agccacgcac ccttgaaagg caccgggtcc ttcttagcat cgtgcttcct |
| | Exemplary nucleic | 61 | gagcaagcct ggcattgcct cacagacctt cctcagagcc gctttcagaa aagcaagctg |
| | acid sequence | 121 | cttctggttg ggcccagacc tgccttgagg agcctgtaga gttaaaaaat gaaccccacg |
| | encoding human | 181 | gatatagcag acaccaccct cgatgaaagc atatacagca attactatct gtatgaaagt |
| | CCR4 | 241 | atccccaagc cttgcaccaa agaaggcatc aaggcatttg gggagctctt cctgccccca |
| | | 301 | ctgtattcct tggtttttgt atttggtctg cttggaaatt ctgtggtggt tctggtcctg |
| | | 361 | ttcaaataca agcggctcag gtccatgact gatgtgtacc tgctcaacct tgccatctcg |
| | | 421 | gatctgctct tcgtgttttc cctccctttt tggggctact atgcagcaga ccagtgggtt |
| | | 481 | tttgggctag gtctgtgcaa gatgatttcc tggatgtact tggtgggctt ttacagtggc |
| | | 541 | atattctttg tcatgctcat gagcattgat agatacctgg caattgtgca cgcggtgttt |
| | | 601 | tccttgaggg caaggacctt gacttatggg gtcatcacca gtttggctac atggtcagtg |
| | | 661 | gctgtgttcg cctcccttcc tggctttctg ttcagcactt gttatactga gcgcaaccat |
| | | 721 | acctactgca aaaccaagta ctctctcaac tccacgacgt ggaaggttct cagctccctg |
| | | 781 | gaaatcaaca ttctcggatt ggtgatcccc ttagggatca tgctgttttg ctactccatg |
| | | 841 | atcatcagga ccttgcagca ttgtaaaaat gagaagaaga acaaggcggt gaagatgatc |
| | | 901 | tttgccgtgg tggtcctctt ccttgggttc tggacacctt acaacatagt gctcttccta |
| | | 961 | gagaccctgg tggagctaga agtccttcag gactgcacct ttgaaagata cttggactat |
| | | 1021 | gccatccagg ccacagaaac tctggctttt gttcactgct gccttaatcc catcatctac |
| | | 1081 | ttttttctgg gggagaaatt tcgcaagtac atcctacagc tcttcaaaac ctgcaggggc |
| | | 1141 | ctttttgtgc tctgccaata ctgtgggctc ctccaaattt actctgctga cacccccagc |
| | | 1201 | tcatcttaca cgcagtccac catggatcat gatctccatg atgctctgta gaaaaatgaa |
| | | 1261 | atggtgaaat gcagagtcaa tgaactttcc acattcagag cttacttaaa attgtatttt |
| | | 1321 | agtaagagat tcctgagcca gtgtcaggag gaaggcttac acccacagtg gaaagacagc |
| | | 1381 | ttctcatcct gcaggcagct ttttctctcc cactagacaa gtccagcctg gcaagggttc |
| | | 1441 | acctgggctg aggcatcctt cctcacacca ggcttgcctg caggcatgag tcagtctgat |
| | | 1501 | gagaactctg agcagtgctt gaatgaagtt gtaggtaata ttgcaaggca aagactattc |
| | | 1561 | ccttctaacc tgaactgatg ggtttctcca gagggaattg cagagtactg gctgatggag |
| | | 1621 | taaatcgcta ccttttgctg tggcaaatgg gccctct |
| 22 | P51679A | 1 | mnptdiadtt ldesiysnyy lyesipkpct kegikafgel flpplyslvf vfgllgnsvv |
| | Exemplary amino | 61 | vlvlfkykrl rsmtdvylln laisdllfvf slpfwgyyaa dqwvfglglc kmiswmylvg |
| | acid sequence for | 121 | fysgiffvml msidrylaiv havfslrart ltygvitsla twsvavfasl pgflfstcyt |
| | human CCR4 | 181 | ernhtycktk yslnsttwkv lssleinilg lviplgimlf cysmiirtlq hcknekknka |
| | precursor | 241 | vkmifavvvl flgfwtpyni vlfletlvel evlqdctfer yldyaiqate tlafvhccln |
| | | 301 | piiyfflgek frkyilqlfk tcrglfvlcq ycgllqiysa dtpsssytqs tmdhdlhdal |
| 23 | NM_001206609.1 | 1 | aatcatccga gaaccttgga gggtggacag tgcccctttt acagatgaga aaactgaggc |
| | Exemplary nucleic | 61 | ttgaagggga gaagcagctg cctctggcgg catggcttct ggctgcagga tgcccatgga |
| | acid sequence | 121 | gttcgtggtg accctaggcc tgtgtctcgg cttcctttgc tgaacttgaa caggaagatg |
| | encoding human | 181 | gcagtggggg ccagtggtct agaaggagat aagatggctg gtgccatgcc tctgcaactc |
| | CLA | 241 | ctcctgttgc tgatcctact gggccctggc aacagcttgc agctgtggga cacctgggca |
| | | 301 | gatgaagccg agaaagcctt gggtcccctg cttgcccggg accggagaca ggccaccgaa |
| | | 361 | tatgagtacc tagattatga tttcctgcca gaaacggagc ctccagaaat gctgaggaac |
| | | 421 | agcactgaca ccactcctct gactgggcct ggaacccctg agtctaccac tgtggagcct |
| | | 481 | gctgcaaggc gttctactgg cctggatgca ggaggggcag tcacagagct gaccacggag |
| | | 541 | ctggccaaca tggggaacct gtccacggat tcagcagcta tggagataca gaccactcaa |
| | | 601 | ccagcagcca cggaggcaca gaccactcaa ccagtgccca cggaggcaca gaccactcca |
| | | 661 | ctggcagcca cagaggcaca gacaactcga ctgacggcca cggaggcaca gaccactcca |
| | | 721 | ctggcagcca cagaggcaca gaccactcca ccagcagcca cggaagcaca gaccactcaa |
| | | 781 | cccacaggcc tggaggcaca gaccactgca ccagcagcca tggaggcaca gaccactgca |
| | | 841 | ccagcagcca tggaagcaca gaccactcca ccagcagcca tggaggcaca gaccactcaa |
| | | 901 | accacagcca tggaggcaca gaccactgca ccagaagcca cggaggcaca gaccactcaa |
| | | 961 | cccacagcca cggaggcaca gaccactcca ctggcagcca tggaggccct gtccacagaa |
| | | 1021 | cccagtgcca cagaggccct gtccatggaa cctactacca aaagaggtct gttcataccc |
| | | 1081 | ttttctgtgt cctctgttac tcacaagggc attcccatgg cagccagcaa tttgtccgtc |
| | | 1141 | aactacccag tgggggcccc agaccacatc tctgtgaagc agtgcctgct ggccatccta |
| | | 1201 | atcttggcgc tggtggccac tatcttcttc gtgtgcactg tggtgctggc ggtccgcctc |
| | | 1261 | tcccgcaagg gccacatgta ccccgtgcgt aattactccc ccaccgagat ggtctgcatc |
| | | 1321 | tcatccctgt tgcctgatgg gggtgagggg ccctctgcca cagccaatgg gggcctgtcc |
| | | 1381 | aaggccaaga gcccgggcct gacgccagag cccagggagg accgtgaggg ggatgacctc |
| | | 1441 | accctgcaca gcttcctccc ttagctcact ctgccatctg ttttggcaag accccacctc |
| | | 1501 | cacgggctct cctgggccac ccctgagtgc ccagacccca ttccacagct ctgggcttcc |
| | | 1561 | tcggagaccc ctggggatgg ggatcttcag ggaaggaact ctggccaccc aaacaggaca |
| | | 1621 | agagcagcct ggggccaagc agacgggcaa gtggagccac ctctttcctc cctccgcgga |
| | | 1681 | tgaagcccag ccacatttca gccgaggtcc aaggcaggag gccatttact tgagacagat |
| | | 1741 | tctctccttt ttcctgtccc ccatcttctc tgggtccctc taacatctcc catggctctc |
| | | 1801 | cccgcttctc ctggtcactg gagtctcctc cccatgtacc caaggaagat ggagctcccc |
| | | 1861 | catcccacac gcactgcact gccattgtct tttggttgcc atggtcacca aacaggaagt |
| | | 1921 | ggacattcta agggaggagt actgaagagt gacggacttc tgaggctgtt tcctgctgct |
| | | 1981 | cctctgactt ggggcagctt gggtcttctt gggcacctct ctgggaaaac ccagggtgag |
| | | 2041 | gttcagcctg tgagggctgg gatgggtttc gtgggcccaa gggcagacct ttctttggga |
| | | 2101 | ctgtgtggac caaggagctt ccatctagtg acaagtgacc cccagctatc gcctcttgcc |
| | | 2161 | ttcccctgtg gccactttcc agggtggact ctgtcttgtt cactgcagta tcccaactgc |
| | | 2221 | aggtccagtg caggcaataa atatgtgatg gacaaacgat agcggaatcc ttcaaggttt |
| | | 2281 | caaggctgtc tccttcaggc agccttcccg gaattctcca tccctcagtg caggatgggg |
| | | 2341 | gctggtcctc agctgtctgc cctcagcccc tggcccccca ggaagcctct ttcatgggct |
| | | 2401 | gttaggttga cttcagtttt gcctcttgga caacaggggg tcttgtacat ccttgggtga |
| | | 2461 | ccaggaaaag ttcaggctat ggggggccaa agggagggct gccccttccc caccagtgac |
| | | 2521 | cactttattc cacttcctcc attacccagt tttggcccac agagtttggt cccccccaaa |
| | | 2581 | cctcggacca atatccctct aaacatcaat ctatcctcct gttaaagaaa aaaaaaaa |
| 24 | NM_003006A | 1 | acacacagcc attgggggtt gctcggatcc gggactgccg cagggggtgc cacagcagtg |
| | Exemplary nucleic | 61 | cctggcagcg tgggctggga ccttgtcact aaagcagaga agccacttct tctgggccca |
| | acid sequence | 121 | cgaggcagct gtcccatgct ctgctgagca cggtggtgcc atgcctctgc aactcctcct |
| | encoding human | 181 | gttgctgatc ctactgggcc ctggcaacag cttgcagctg tgggacacct gggcagatga |
| | CLA | 241 | agccgagaaa gccttgggtc ccctgcttgc ccgggaccgg agacaggcca ccgaatatga |
| | | 301 | gtacctagat tatgatttcc tgccagaaac ggagcctcca gaaatgctga ggaacagcac |
| | | 361 | tgacaccact cctctgactg ggcctggaac ccctgagtct accactgtgg agcctgctgc |
| | | 421 | aaggcgttct actggcctgg atgcaggagg ggcagtcaca gagctgacca cggagctggc |
| | | 481 | caacatgggg aacctgtcca cggattcagc agctatggag atacagacca ctcaaccagc |
| | | 541 | agccacggag gcacagacca ctcaaccagt gcccacggag gcacagacca ctccactggc |
| | | 601 | agccacagag gcacagacaa ctcgactgac ggccacggag gcacagacca ctccactggc |
| | | 661 | agccacagag gcacagacca ctccaccagc agccacggaa gcacagacca ctcaacccac |
| | | 721 | aggcctggag gcacagacca ctgcaccagc agccatggag gcacagacca ctgcaccagc |
| | | 781 | agccatggaa gcacagacca ctccaccagc agccatggag gcacagacca ctcaaaccac |
| | | 841 | agccatggag gcacagacca ctgcaccaga agccacggag gcacagacca ctcaacccac |
| | | 901 | agccacggag gcacagacca ctccactggc agccatggag gccctgtcca cagaacccag |
| | | 961 | tgccacagag gccctgtcca tggaacctac taccaaaaga ggtctgttca tacccttttc |
| | | 1021 | tgtgtcctct gttactcaca agggcattcc catggcagcc agcaatttgt ccgtcaacta |
| | | 1081 | cccagtgggg gccccagacc acatctctgt gaagcagtgc ctgctggcca tcctaatctt |
| | | 1141 | ggcgctggtg gccactatct tcttcgtgtg cactgtggtg ctggcggtcc gcctctcccg |
| | | 1201 | caagggccac atgtaccccg tgcgtaatta ctcccccacc gagatggtct gcatctcatc |
| | | 1261 | cctgttgcct gatgggggtg aggggccctc tgccacagcc aatgggggcc tgtccaaggc |
| | | 1321 | caagagcccg ggcctgacgc cagagcccag ggaggaccgt gagggggatg acctcaccct |
| | | 1381 | gcacagcttc ctcccttagc tcactctgcc atctgttttg gcaagacccc acctccacgg |
| | | 1441 | gctctcctgg gccacccctg agtgcccaga ccccattcca cagctctggg cttcctcgga |
| | | 1501 | gacccctggg gatggggatc ttcagggaag gaactctggc cacccaaaca ggacaagagc |
| | | 1561 | agcctggggc caagcagacg ggcaagtgga gccacctctt tcctccctcc gcggatgaag |
| | | 1621 | cccagccaca tttcagccga ggtccaaggc aggaggccat ttacttgaga cagattctct |
| | | 1681 | cctttttcct gtcccccatc ttctctgggt ccctctaaca tctcccatgg ctctccccgc |
| | | 1741 | ttctcctggt cactggagtc tcctccccat gtacccaagg aagatggagc tcccccatcc |
| | | 1801 | cacacgcact gcactgccat tgtcttttgg ttgccatggt caccaaacag gaagtggaca |
| | | 1861 | ttctaaggga ggagtactga agagtgacgg acttctgagg ctgtttcctg ctgctcctct |
| | | 1921 | gacttggggc agcttgggtc ttcttgggca cctctctggg aaaacccagg gtgaggttca |
| | | 1981 | gcctgtgagg gctgggatgg gtttcgtggg cccaagggca gacctttctt tgggactgtg |
| | | 2041 | tggaccaagg agcttccatc tagtgacaag tgacccccag ctatcgcctc ttgccttccc |
| | | 2101 | ctgtggccac tttccagggt ggactctgtc ttgttcactg cagtatccca actgcaggtc |
| | | 2161 | cagtgcaggc aataaatatg tgatggacaa acgatagcgg aatccttcaa ggtttcaagg |
| | | 2221 | ctgtctcctt caggcagcct tcccggaatt ctccatccct cagtgcagga tgggggctgg |
| | | 2281 | tcctcagctg tctgccctca gcccctggcc ccccaggaag cctctttcat gggctgttag |
| | | 2341 | gttgacttca gttttgcctc ttggacaaca gggggtcttg tacatccttg ggtgaccagg |
| | | 2401 | aaaagttcag gctatggggg gccaaaggga gggctgcccc ttccccacca gtgaccactt |
| | | 2461 | tattccactt cctccattac ccagttttgg cccacagagt ttggtccccc ccaaacctcg |
| | | 2521 | gaccaatatc cctctaaaca tcaatctatc ctcctgttaa agaaaaaaaa aaa |
| 25 | NP_001193538.1 | 1 | mavgasgleg dkmagamplq lllllillgp gnslqlwdtw adeaekalgp llardrrqat |
| | Exemplary amino | 61 | eyeyldydfl peteppemlr nstdttpltg pgtpesttve paarrstgld aggavteltt |
| | acid sequence for | 121 | elanmgnlst dsaameiqtt qpaateaqtt qpvpteaqtt plaateaqtt rltateaqtt |
| | human CLA | 181 | plaateaqtt ppaateaqtt qptgleaqtt apaameaqtt apaameaqtt ppaameaqtt |
| | precursor | 241 | qttameaqtt apeateaqtt qptateaqtt plaamealst epsatealsm epttkrglfi |
| | | 301 | pfsvssvthk gipmaasnls vnypvgapdh isvkqcllai lilalvatif fvctvvlavr |
| | | 361 | lsrkghmypv rnysptemvc issllpdgge gpsatanggl skakspgltp epredregdd |
| | | 421 | ltlhsflp |
| 26 | NP_002997.2 | 1 | mplqllllli llgpgnslql wdtwadeaek algpllardr rqateyeyld ydflpetepp |
| | Exemplary amino | 61 | emlrnstdtt pltgpgtpes ttvepaarrs tgldaggavt elttelanmg nlstdsaame |
| | acid sequence for | 121 | iqttqpaate aqttqpvpte aqttplaate aqttrltate aqttplaate aqttppaate |
| | human CLA | 181 | aqttqptgle aqttapaame aqttapaame aqttppaame aqttqttame aqttapeate |
| | precursor | 241 | aqttqptate aqttplaame alstepsate alsmepttkr glfipfsvss vthkgipmaa |
| | | 301 | snlsvnypvg apdhisvkqc llaililalv atiffvctvv lavrlsrkgh mypvrnyspt |
| | | 361 | emvcissllp dggegpsata ngglskaksp gltpepredr egddltlhsf lp |

4.4. Chimeric Antigen Receptors

When the cells provided herein are T lymphocytes which comprise homing receptors, as described above, such T lymphocytes can, in certain embodiments, comprise chimeric antigen receptors (CARs), which are artificial membrane-bound proteins that direct a T lymphocyte to an antigen, and stimulate the T lymphocyte to kill a cell displaying the antigen. See, e.g., Eshhar, U.S. Pat. No. 7,741,465. At a minimum, the CAR comprises an extracellular domain that binds to an antigen, e.g., an antigen on a cell, a transmembrane domain, and an intracellular (cytoplasmic) signaling domain that transmits a primary activation signal to an immune cell. All other conditions being satisfied, when the CAR is expressed on the surface of, e.g., a T lymphocyte, for example, a primary T lymphocyte, and the extracellular domain of the CAR binds to an antigen, the intracellular signaling domain transmits a signal to the T lymphocyte to activate and/or proliferate, and, if the antigen is present on a cell surface, to kill the cell expressing the antigen. Because T lymphocytes require two signals, a primary activation signal and a costimulatory signal, in order to maximally activate, typically CARs also comprise a costimulatory domain such that binding of the antigen to the extracellular domain results in transmission of both a primary activation signal and a costimulatory signal.

4.4.1. General CAR Structure Intracellular Domain

In certain embodiments, the intracellular domain of the CAR is or comprises an intracellular domain or motif of a protein that is expressed on the surface of T lymphocytes and triggers activation and/or proliferation of said T lymphocytes. Such a domain or motif is able to transmit a primary antigen-binding signal that is necessary for the activation of a T lymphocyte in response to the antigen's binding to the CAR's extracellular portion. Typically, this domain or motif comprises, or is, an ITAM (immunoreceptor tyrosine-based activation motif). ITAM-containing polypeptides suitable for CARs include, for example, the zeta CD3 chain (CD3ζ) or ITAM-containing portions thereof. In a specific embodiment, the intracellular domain is a CD3ζ intracellular signaling domain. In other specific embodiments, the intracellular domain is from a lymphocyte receptor chain, a TCR/CD3 complex protein, an Fc receptor subunit or an IL-2 receptor subunit.

In certain embodiments, the CAR additionally comprises one or more co-stimulatory domains or motifs, e.g., as part of the intracellular domain of the polypeptide. The one or more co-stimulatory domains or motifs can be, or comprise, one or more of a co-stimulatory CD27 polypeptide sequence, a co-stimulatory CD28 polypeptide sequence, a co-stimulatory OX40 (CD134) polypeptide sequence, a co-stimulatory 4-1BB (CD137) polypeptide sequence, or a co-stimulatory inducible T-cell costimulatory (ICOS) polypeptide sequence, or other costimulatory domain or motif.

The transmembrane region can be any transmembrane region that can be incorporated into a functional CAR, typically a transmembrane region from a CD4 or a CD8 molecule.

4.4.2. CAR Extracellular Domain

The extracellular domain of the polypeptide binds to an antigen of interest. In certain embodiments of any of the polypeptides described herein, the extracellular domain comprises a receptor, or a portion of a receptor, that binds to said antigen. The extracellular domain may be, e.g., a receptor, or a portion of a receptor, that binds to said antigen. In certain embodiments, the extracellular domain comprises, or is, an antibody or an antigen-binding portion thereof. In specific embodiments, the extracellular domain comprises, or is, a single-chain Fv domain. The single-chain Fv domain can comprise, for example, a V_{L} linked to V_{H} by a flexible linker, wherein said V_{L} and V_{H} are from an antibody that binds said antigen.

The antigen to which the extracellular domain of the polypeptide binds can be any antigen of interest, e.g., can be an antigen on a tumor cell. The tumor cell may be, e.g., a cell in a solid tumor, or a cell of a blood cancer. The antigen can be any antigen that is expressed on a cell of any tumor or cancer type, e.g., cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

In certain embodiments, the antigen is a tumor-associated antigen or a tumor-specific antigen. In various specific embodiments, without limitation, the tumor-associated antigen or tumor-specific antigen is Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD19, CD34, CD45, CD99, CD117, chromogranin, cytokeratin, desmin, glial fibrillary acidic protein (GFAP), gross cystic disease fluid protein (GCDFP-15), HMB-45 antigen, high molecular weight melanoma-associated antigen (HMW-MAA), protein melan-A (melanoma antigen recognized by T lymphocytes; MART-1), myo-D1, muscle-specific actin (MSA), neurofilament, neuron-specific enolase (NSE), placental alkaline phosphatase, synaptophysis, thyroglobulin, thyroid transcription factor-1, the dimeric form of the pyruvate kinase isoenzyme type M2 (tumor M2-PK), an abnormal ras protein, or an abnormal p53 protein.

In certain embodiments, the TAA or TSA is a cancer/testis (CT) antigen, e.g., BAGE, CAGE, CTAGE, FATE, GAGE, HCA661, HOM-TES-85, MAGEA, MAGEB, MAGEC, NA88, NY-ESO-1, NY-SAR-35, OY-TES-1, SPANXB1, SPA17, SSX, SYCP1, or TPTE.

In certain other embodiments, the TAA or TSA is a carbohydrate or ganglioside, e.g., fuc-GM1, GM2 (oncofetal antigen-immunogenic-1; OFA-1-1); GD2 (OFA-1-2), GM3, GD3, and the like.

In certain other embodiments, the TAA or TSA is alpha-actinin-4, Bage-1, BCR-ABL, Bcr-Abl fusion protein, beta-catenin, CA 125, CA 15-3 (CA 27.29\BCAA), CA 195, CA 242, CA-50, CAM43, Casp-8, cdc27, cdk4, cdkn2a, CEA, coa-1, dek-can fusion protein, EBNA, EF2, Epstein Barr virus antigens, ETV6-AML1 fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, triosephosphate isomerase, Gage 3,4,5,6,7, GnTV, Herv-K-mel, Lage-1, NA-88, NY-Eso-1/Lage-2, SP17, SSX-2, TRP2-Int2, gp100 (Pmel 17), tyrosinase, TRP-1, TRP-2, MAGE-1, MAGE-3, RAGE, GAGE-1, GAGE-2, p15(58), RAGE, SCP-1, Hom/Mel-40, PRAME, p53, H-Ras, HER-2/neu, E2A-PRL, H4-RET, IGH-IGK, MYL-RAR, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, 13-Catenin, Mum-1, p16, TAGE, PSMA, CT7, telomerase, 43-9F, 5T4, 791Tgp72, 13HCG, BCA225, BTAA, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90, TAAL6, TAG72, TLP, TPS, CD19, CD22, CD27, CD30, CD70, GD2 (ganglioside G2), EGFRvIII (epidermal growth factor variant 111), sperm protein 17 (Sp17), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B. Other tumor-associated and tumor-specific antigens are known to those in the art.

Antibodies, and scFvs, that bind to TSAs and TAAs are known in the art, as are nucleotide sequences that encode them.

In certain specific embodiments, the antigen is an antigen not considered to be a TSA or a TAA, but which is nevertheless associated with tumor cells, or damage caused by a tumor. In certain embodiments, for example, the antigen is, e.g., a growth factor, cytokine or interleukin, e.g., a growth factor, cytokine, or interleukin associated with angiogenesis or vasculogenesis. Such growth factors, cytokines, or interleukins can include, e.g., vascular endothelial growth factor (VEGF), basic fibroblast growth factor (bFGF), platelet-derived growth factor (PDGF), hepatocyte growth factor (HGF), insulin-like growth factor (IGF), or interleukin-8 (IL-8). Tumors can also create a hypoxic environment local to the tumor. As such, in other specific embodiments, the antigen is a hypoxia-associated factor, e.g., HIF-1α, HIF-1β, HIF-2α, HIF-2β, HIF-3α, or HIF-3β. Tumors can also cause localized damage to normal tissue, causing the release of molecules known as damage associated molecular pattern molecules (DAMPs; also known as alarmins). In certain other specific embodiments, therefore, the antigen is a DAMP, e.g., a heat shock protein, chromatin-associated protein high mobility group box 1 (HMGB1), S100A8 (MRP8, calgranulin A), S100A9 (MRP14, calgranulin B), serum amyloid A (SAA), or can be a deoxyribonucleic acid, adenosine triphosphate, uric acid, or heparin sulfate.

In a specific embodiment, in which the cancer a gastrointestinal cancer, for example, liver cancer, stomach cancer, esophageal cancer, gallbladder cancer, colorectal cancer, anal cancer, or pancreatic cancer, the antigen is an antigen specific for or associated with a gastrointestinal cancer. In a specific embodiment, T lymphocytes provided herein express a gastrointestinal homing receptor and also express a CAR with an extracellular domain that binds to an antigen associated with a gastrointestinal cancer. In a specific embodiment, the extracellular domain of the CAR binds CEA. In other specific embodiments, the extracellular domain of the CAR binds Her2, CA242, MUC1, CA125, or CA19-9.

In a specific embodiment, in which the cancer is a skin cancer, for example, melanoma, squamous cell carcinoma, or basal cell carcinoma, the antigen is an antigen specific for or associated with a skin cancer. In a specific embodiment, T lymphocytes provided herein express a skin homing receptor and also express a CAR with an extracellular domain that binds to an antigen associated with a skin cancer. In a specific embodiment, the extracellular domain of the CAR binds HMW-MAA. In other specific embodiments, the extracellular domain of the CAR binds Her2, GD2, GD3, CEA, or SPAG9.

In certain embodiments of the polypeptides described herein, the extracellular domain is joined to said transmembrane domain by a linker, spacer or hinge polypeptide sequence, e.g., a sequence from CD28.

4.5. Isolated Nucleic Acids

In one embodiment, provided herein are polynucleotide sequences that encode the polypeptides provided herein (e.g., chimeric receptors and homing receptors). The polynucleotides may be contained within any polynucleotide vector suitable for the transformation of immune cells, e.g., T lymphocytes. For example, T lymphocytes may be transformed using synthetic vectors, lentiviral or retroviral vectors, autonomously replicating plasmids, a virus (e.g., a retrovirus, lentivirus, adenovirus, or herpes virus), or the like, containing polynucleotides encoding the first and second polypeptides (e.g., chimeric receptors). Lentiviral vectors suitable for transformation of T lymphocytes include, but are not limited to, e.g., the lentiviral vectors described in U.S. Pat. Nos. 5,994,136; 6,165,782; 6,428,953; 7,083,981; and 7,250,299, the disclosures of which are hereby incorporated by reference in their entireties. HIV vectors suitable for transformation of T lymphocytes include, but are not limited to, e.g., the vectors described in U.S. Pat. No. 5,665,577, the disclosure of which is hereby incorporated by reference in its entirety.

Nucleic acids useful in the production of the polypeptides provided herein, e.g., within a T lymphocyte, include DNA, RNA, or nucleic acid analogs. Nucleic acid analogs can be modified at the base moiety, sugar moiety, or phosphate backbone, and can include deoxyuridine substitution for deoxythymidine, 5-methyl-2′-deoxycytidine or 5-bromo-2′-deoxycytidine substitution for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chain. 4:5-23. In addition, the deoxyphosphate backbone can be replaced with, for example, a phosphorothioate or phosphorodithioate backbone, a phosphoroamidite, or an alkyl phosphotriester backbone.

A nucleic acid encoding a polypeptide provided herein may be introduced into host cells as part of a vector, such as, e.g., an expression vector. In addition, a polypeptide provided herein may be produced by transfecting a host cell with a nucleic acid encoding such a polypeptide, and such nucleic acid may be part of a vector. In a specific embodiment, the vector is an expression vector that is capable of directing the expression of a nucleic acid encoding a polypeptide provided herein. Non-limiting examples of expression vectors include, but are not limited to, plasmids and viral vectors, such as replication defective retroviruses, adenoviruses, adeno-associated viruses, Newcastle disease virus, vaccinia virus and baculoviruses. Standard molecular biology techniques may be used to introduce a nucleic acid encoding a polypeptide provided herein into an expression vector.

An expression vector comprises a nucleic acid encoding a polypeptide provided herein in a form suitable for expression of the nucleic acid in a host cell or non-human subject. In a specific embodiment, an expression vector includes one or more regulatory sequences, selected on the basis of the host cells to be used for expression, which is operably linked to the nucleic acid to be expressed. Within an expression vector, “operably linked” is intended to mean that a nucleic acid of interest is linked to the regulatory sequence(s) in a manner which allows for expression of the nucleic acid (e.g., in an in vitro transcription/translation system or in a host cell when the vector is introduced into the host cell). Regulatory sequences include promoters, enhancers and other expression control elements (e.g., polyadenylation signals). Regulatory sequences include those which direct constitutive expression of a nucleic acid in many types of host cells, those which direct expression of the nucleic acid only in certain host cells (e.g., tissue-specific regulatory sequences), and those which direct the expression of the nucleic acid upon stimulation with a particular agent (e.g., inducible regulatory sequences). It will be appreciated by those skilled in the art that the design of the expression vector can depend on such factors as, e.g., the choice of the host cell to be transformed, the level of expression of protein desired, etc.

An expression vector can be introduced into host cells via conventional transformation or transfection techniques. Such techniques include, but are not limited to, calcium phosphate or calcium chloride co-precipitation, DEAE-dextran-mediated transfection, lipofection, and electroporation. Suitable methods for transforming or transfecting host cells can be found in Sambrook et al., 1989, Molecular Cloning—A Laboratory Manual, 2nd Edition, Cold Spring Harbor Press, New York, and other laboratory manuals. In certain embodiments, a host cell is transiently transfected with an expression vector containing a nucleic acid encoding a polypeptide provided herein. In other embodiments, a host cell is stably transfected with an expression vector containing a nucleic acid encoding a polypeptide provided herein.

4.6. Cells

Non-limiting examples of cells in which the homing receptors may be used include, but are not limited to, natural killer (NK) cells, dendritic cells (DC), placental stem cells (e.g., the placental stem cells disclosed in U.S. Pat. Nos. 7,468,276; 8,057,788 and 8,202,703, the disclosures of which are hereby incorporated by reference in their entireties), mesenchymal-like stem cells from umbilical cord blood, placental blood, peripheral blood, bone marrow, dental pulp, adipose tissue, osteochondral tissue, and the like; embryonic stem cells, embryonic germ cells, neural crest stem cells, neural stem cells, and differentiated cells (e.g., fibroblasts, etc.). The homing receptors may also be used in tumor cell lines, e.g., for animal model experimental purposes.

In a specific embodiment, the cells comprising the polypeptides provided herein are T lymphocytes. The T lymphocytes comprising the polypeptides provided herein may be naive T lymphocytes or MHC-restricted T lymphocytes. In certain embodiments, the T lymphocytes are tumor infiltrating lymphocytes (TILs). In certain embodiments, the T lymphocytes have been isolated from a tumor biopsy, or have been expanded from T lymphocytes isolated from a tumor biopsy. In certain other embodiments, the T lymphocytes have been isolated from, or are expanded from T lymphocytes expanded from, peripheral blood, cord blood, or lymph.

In certain embodiments, the immune cells, e.g., T lymphocytes, used in the present methods are autologous to an individual to whom the T lymphocytes are to be administered. In certain embodiments, the T lymphocytes are allogeneic to an individual to whom the T lymphocytes are to be administered. Where allogeneic T lymphocytes are used to prepare T lymphocytes, it is preferable to select T lymphocytes that will reduce the possibility of graft-versus-host disease (GVHD) in the individual. For example, in certain embodiments, virus-specific T lymphocytes are selected for preparation of T lymphocytes; such lymphocytes will be expected to have a greatly reduced native capacity to bind to, and thus become activated by, any recipient antigens. In certain embodiments, recipient-mediated rejection of allogeneic T lymphocytes can be reduced by co-administration to the host of one or more immunosuppressive agents, e.g., cyclosporine, tacrolimus, sirolimus, cyclophosphamide, or the like.

In one embodiment, T lymphocytes are obtained from an individual, optionally then expanded, and then transformed with a polynucleotide encoding one or more homing receptors, and optionally one or more polynucleotides encoding one or more CAR(s), and optionally then expanded. In another embodiment, T lymphocytes are obtained from an individual, optionally then expanded, and then transformed with a polynucleotide encoding one or more homing receptors, and optionally one or more polynucleotides encoding one or more CAR(s), and optionally then expanded. Cells containing any of the polynucleotide may be selected using one or more selectable markers.

In certain embodiments, any of the T lymphocytes provided herein express or comprise native TCR proteins, e.g., TCR-α and TCR-β that are capable of forming native TCR complexes. In certain other embodiments, either or both of the native genes encoding TCR-α and TCR-β in the T lymphocytes are modified to be non-functional, e.g., a portion or all are deleted, a mutation is inserted, etc.

In certain embodiments, any of the T lymphocytes provided herein are isolated from a tumor lesion, e.g., are tumor-infiltrating lymphocytes; such T lymphocytes are expected to be specific for a TSA or TAA.

T lymphocytes, and T lymphocytes comprising a polypeptide comprising a CD3ζ signaling domain and a CD28 co-stimulatory domain can be expanded using antibodies to CD3 and CD28, e.g., antibodies attached to beads, or to the surface of a cell culture plate; see, e.g., U.S. Pat. Nos. 5,948,893; 6,534,055; 6,352,694; 6,692,964; 6,887,466; and 6,905,681.

In certain embodiments, the antigen and/or antibody can exist free in the medium in which the T lymphocytes are cultured, or either or both can be attached to a solid support, e.g., tissue culture plastic surface, beads, or the like.

4.7. Methods of Using Cells Comprising Homing Receptors

In one aspect, the cells, e.g., T lymphocytes, provided herein that comprise one or more homing receptors and optionally one or more CARs, as described elsewhere herein, are used to treat an individual having one or more types of cells desired to be targeted by the cells described herein, e.g., one or more types of cells to be killed. In certain embodiments, the cells to be killed are cancer cells, e.g., tumor cells. In specific embodiments, the cancer cells are cells of a solid tumor. In specific embodiments, the cells are cells of a lymphoma, a lung cancer, a breast cancer, a prostate cancer, an adrenocortical carcinoma, a thyroid carcinoma, a nasopharyngeal carcinoma, a melanoma, e.g., a malignant melanoma, a skin carcinoma, a colorectal carcinoma, a desmoid tumor, a desmoplastic small round cell tumor, an endocrine tumor, an Ewing sarcoma, a peripheral primitive neuroectodermal tumor, a solid germ cell tumor, a hepatoblastoma, a neuroblastoma, a non-rhabdomyosarcoma soft tissue sarcoma, an osteosarcoma, a retinoblastoma, a rhabdomyosarcoma, a Wilms tumor, a glioblastoma, a myxoma, a fibroma, a lipoma, or the like. In more specific embodiments, said lymphoma can be chronic lymphocytic leukemia (small lymphocytic lymphoma), B-cell prolymphocytic leukemia, lymphoplasmacytic lymphoma, Waldenström macroglobulinemia, splenic marginal zone lymphoma, plasma cell myeloma, plasmacytoma, extranodal marginal zone B cell lymphoma, MALT lymphoma, nodal marginal zone B cell lymphoma, follicular lymphoma, mantle cell lymphoma, diffuse large B cell lymphoma, mediastinal (thymic) large B cell lymphoma, intravascular large B cell lymphoma, primary effusion lymphoma, Burkitt's lymphoma, T lymphocyte prolymphocytic leukemia, T lymphocyte large granular lymphocytic leukemia, aggressive NK cell leukemia, adult T lymphocyte leukemia/lymphoma, extranodal NK/T lymphocyte lymphoma, nasal type, enteropathy-type T lymphocyte lymphoma, hepatosplenic T lymphocyte lymphoma, blastic NK cell lymphoma, mycosis fungoides, Sezary syndrome, primary cutaneous anaplastic large cell lymphoma, lymphomatoid papulosis, angioimmunoblastic T lymphocyte lymphoma, peripheral T lymphocyte lymphoma (unspecified), anaplastic large cell lymphoma, Hodgkin lymphoma, or a non-Hodgkin lymphoma.

In certain embodiments, the modified cells, e.g., modified T lymphocytes described herein are administered to a subject in need thereof, such that the combination of homing receptors selected is compatible with the patient population (or subpopulation) in which the cells, e.g., T lymphocytes, have been administered. For example, the combination of homing receptors is chosen based on the type of tumor or cancer present in the patient.

In specific embodiments, T lymphocytes expressing gastrointestinal homing receptors are administered to patients having a gastrointestinal tumor or cancer. In certain embodiments, the gastrointestinal tumor or cancer is esophageal cancer, stomach cancer, liver cancer, gallbladder cancer, pancreatic cancer, colorectal cancer, or anal cancer. In a specific embodiment, said T cells further comprise a CAR with an extracellular domain that binds to an antigen associated with a gastrointestinal tumor or cancer.

In other specific embodiments, T lymphocytes expressing skin homing receptors are administered to patients having a skin tumor or cancer. In certain embodiments, the skin tumor or cancer is melanoma, basal cell carcinoma, or squamous cell carcinoma. In a specific embodiment, said T cells further comprise a CAR with an extracellular domain that binds to an antigen associated with a skin tumor or cancer. In specific embodiments, the T lymphocytes comprising a homing receptor also comprise a CAR with an extracellular domain that binds to an antigen expressed in the tumor or cancer cells of the patient.

Efficacy of the cells, e.g., T lymphocytes, after administration to an individual having a disease or disorder remediable by such cells, e.g., T lymphocytes, e.g., an individual having cancer, can be assessed by one or more criteria, specific to the particular disease or disorder, known to those of ordinary skill in the art, to be indicative of progress of the disease or disorder. Generally, administration of the cells to such an individual is effective when one or more of said criteria detectably, e.g., significantly, moves from a disease state value or range to, or towards, a normal value or range.

In certain embodiments, the cells, e.g., T lymphocytes, are formulated in a pharmaceutically-acceptable solution. In preferred embodiments, the pharmaceutically-acceptable solution is suitable for the delivery of living cells. In specific embodiments, the pharmaceutically-acceptable solution is, for example, saline solution (such as Ringer's solution), gelatins, carbohydrates (e.g., lactose, amylose, starch, or the like), fatty acid esters, hydroxymethylcellulose, or polyvinyl pyrolidine. In more specific embodiments, the pharmaceutically-acceptable solution is sterilized prior to addition of the cells. In other more specific embodiments, the pharmaceutically-acceptable solution may be mixed with auxiliary agents such as lubricants, preservatives, stabilizers, emulsifiers, salts for influencing osmotic pressure, buffers, and coloring. Pharmaceutical carriers suitable for use in formulating the cells are known in the art and are described, for example, in WO 96/05309.

In certain embodiments, the cells, e.g., T lymphocytes, are formulated into individual doses, wherein said individual doses comprise at least, at most, or about 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ T lymphocytes. In certain embodiments, the cells, e.g., T lymphocytes, are formulated into individual doses, wherein said individual does comprise a range from 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, or 5×10¹⁰ to 1×10¹¹ T lymphocytes. In certain embodiments, the cells are formulated for intravenous, intra-arterial, parenteral, intramuscular, subcutaneous, intrathecal, or intraocular administration, or administration within a particular organ or tissue.

### 5. EXAMPLES

### 5.1. Example 1: Generation of Murine CXCR5⁺ CAR T Cells and In Vivo Study

T lymphocytes are obtained from the spleen of B6 Thy 1.1 mice. A lentiviral construct comprising a chimeric antigen receptor (CAR) comprising humanized mouse anti-human CEA-scFv or anti-human HER2-scFv and mouse co-stimulation intracellular domain and CD3ζ is generated. Validation of the CAR T cells is done via phenotypic characterization and functional evaluation via a mouse CXCL13 chemotaxis assay.

For the in vivo assay, the activities of murine CXCR5+ CAR T cells are compared to the activities of CXCR5− CAR T cells and non-transduced T cells. Human CEA (or HER2)− transgenic mice that can spontaneously develop CEA+ gastrointestinal tumors (or HER2+ mammary tumors) are used as subjects. Adoptive transfer of the cells is accomplished with one or more doses by intravenous administration.

To examine the location of adoptively transferred CAR T cells in draining lymph nodes, (mesenteric lymph nodes for CEA transgenic mice or axillary lymph nodes for HER2 transgenic mice), a readout assay is done via draining lymph node immunohistochemistry. Animals are sacrificed and the draining lymph nodes are identified. Lymph nodes are grasped with curved forceps and pulled free of attached tissue. Lymph nodes are embedded in optimal cutting temperature compound and frozen on dry ice, and then stored at −80° C. Cryosections are cut, air-dried, and fixed in acetone. Sections are pre-incubated with rabbit (or goat) serum at room temperature, and subsequently incubated with primary antibodies (rat anti-mouse CD3, CD8, CD4, biotinylated mouse anti-mouse Thy-1.1, biotinylated goat-anti-human IgG (H+L)), and biotinylated rabbit (or goat) anti-rat IgG (mouse adsorbed) followed by avidin-peroxidase (or fluorochrome) conjugate. Then sections are incubated with substrate of peroxidase for color development. The images of immunohistochemistry staining results are acquired using confocal microscopy. Donor T cells are identified via anti-mouse Thy1.1 and CAR T cells are identified with anti-human IgG.

To examine the antigen-specific immune response establishment in the draining lymph nodes, a readout assay is done via flow cytometry of CAR T cells isolated from draining lymph nodes. Phenotypic characterization of surface markers Thy 1.1, anti-human IgG, CXCR5, CD69, and HLA-DR is performed. CD69 is a marker for activated T cells. HLA-DR is a marker for antigen-presenting cells. Functional evaluation is done to assay proliferation, Ki67 expression, and numerization of CAR T cells. Cytokine production is evaluated by intracellular cytokine staining upon stimulation.

To examine tumor killing activity of CAR T cells in a tumor site, tumor volume and survival are assayed. Volumes of tumors are determined in vivo by external caliper. The greatest longitudinal diameter (length) and the greatest transverse diameter (width) are determined. Tumor volume based on caliper measurements are calculated by the modified ellipsoidal formula: tumor volume=½(length×width²).

### 5.2. Example 2: Generation of Human CXCR5⁺ CAR T Cells

T lymphocytes are obtained from human PBMCs. A lentiviral construct comprising a chimeric antigen receptor (CAR) composed of humanized mouse anti-human CEA-scFv or anti-human HER2-scFv and human co-stimulation intracellular domain and CD3ζ is generated and used to transduce the T cells to create a population of CEA-specific CAR T cells. The CAR T cells are further transduced with lentiviral vectors containing nucleic acid sequences encoding human CXCR5; CAR T cells expressing CXCR5 are selected for further study. Validation of the CAR T cells is done via phenotypic characterization and functional evaluation via a human CXCL13 chemotaxis assay.

### 5.3. Example 3: Generation of Murine Gastrointestinal Homing CAR T Cells and In Vivo Study

T lymphocytes are obtained from the spleen of mice. CAR T cells are generated using a lentiviral construct, as described above, containing a nucleotide sequence encoding a CAR comprising a humanized mouse anti-human CEA-scFv and mouse co-stimulation intracellular domain and CD3ζ. The gastrointestinal homing receptors are introduced via lentiviral transduction of mouse CCR9 and α4β7 nucleic acid sequences. Retinoic acid treatment is performed during T cell activation. Validation of the CAR T cells is done via phenotypic characterization and functional evaluation via a mouse TECK (thymus expressed chemokine, or CCL25) chemotaxis assay (Binger, et al., 2009, Experimental Cell Research 315:1468-79). The chemotactic response to TECK is assessed using 5 μm pore-size polycarbonate membranes in 96-multiwell format chemotaxis plates. Lower wells containing TECK in deprivation medium are separated from the upper wells by the membrane. The T cells, in deprivation medium, are seeded into the upper wells and incubated at 37° C. for 20 hours. Non-migrated cells left on top of the membrane are removed. Migrated cells are fixed, stained, and enumerated microscopically or are enumerated via flow cytometry with counting beads. Negative controls are performed with deprivation medium without chemokine, and positive controls are performed with medium for cell culturing containing 10% FBS.

For an in vivo assay, human CEA-transgenic mice that can spontaneously develop CEA+ gastrointestinal tumors are used as subjects. Adoptive transfer of the cells is accomplished with one or more doses by intravenous administration.

To examine the location of adoptively transferred CAR T cells in established gastrointestinal tract tumors, a readout assay is done via gastrointestinal tract immunohistochemistry. Animals are sacrificed and the gastrointestinal tracts are removed. The small intestine is sectioned into 4-5 parts before immersion fixation. Sections are pre-incubated with rabbit (or goat) serum at room temperature, and subsequently incubated with primary antibodies (rat anti-mouse CD3, biotinylated mouse anti-mouse Thy-1.1, biotinylated goat-anti-human IgG (H+L)), and biotinylated rabbit (or goat) anti-rat IgG (mouse adsorbed) followed by avidin-peroxidase (or fluorochrome) conjugate. Then sections are incubated with substrate of peroxidase for color development. The images of immunohistochemistry staining results are acquired using microscopy. Donor T cells are identified via anti-mouse Thy1.1 and CAR T cells are identified with anti-human IgG. qRT-PCR or PCR of gastrointestinal tract for detection of migrated CAR T cells in the tissue is also performed.

To examine the antigen-specific immune response establishment in the gastrointestinal tract, a readout assay is done via flow cytometry of CAR T cells isolated from the gastrointestinal tract. Phenotypic characterization of surface markers Thy 1.1, anti-human IgG, CCR9, α4β7, CD69, and HLA-DR is performed. Functional evaluation is done to assay proliferation, Ki67 expression, and numerization of CAR T cells. Cytokine production is evaluated by intracellular cytokine staining upon stimulation.

To examine tumor killing activity of CAR T cells in a tumor site, tumor volume and survival are assayed.

### 5.4. Example 4: Generation of Human Gastrointestinal Homing CAR T Cells

T lymphocytes are obtained from human PBMCs. Generation of CAR T cells is performed using a CAR comprising humanized mouse anti-human CEA-scFv and mouse co-stimulation intracellular domain and CD3ζ. The CAR T cells are further transduced with lentiviral vectors containing nucleic acid sequences encoding human CCR9 and α4β7; CAR T cells expressing both of these proteins are selected for further study. During subsequent T cell activation, the CAR T cells are contacted with retinoic acid such that the level of the CCR9 and α4β7 are increased as compared to CAR T cells expressing these two proteins, but not contacted with retinoic acid. Validation of the CAR T cells is done via phenotypic characterization and functional evaluation via a human TECK chemotaxis assay.

### 5.5. Example 5: Generation of Mouse Skin Homing CAR T Cells and In Vivo Study

T lymphocytes are obtained from mouse spleen. Generation of CAR T cells is performed using a CAR comprising humanized mouse anti-human HMW-MAA-scFv and mouse co-stimulation intracellular domain and CD3ζ. The CAR T cell s are further transduced with lentiviral vectors containing nucleic acid sequences encoding human CCR10 or CCR4, and/or CLA; CAR T cells expressing both of these proteins are selected for further study. Validation of the CAR T cells is done via phenotypic characterization and functional evaluation via a mouse CCL27 chemotaxis assay. The chemotactic response to CCL27 is assessed using 5 μm pore-size polycarbonate membranes in 96-multiwell format chemotaxis plates. Lower wells containing CCL27 in deprivation medium are separated from the upper wells by the membrane. The T cells, in deprivation medium, are seeded into the upper wells and incubated at 37° C. for 20 hours. Non-migrated cells left on top of the membrane are removed. Migrated cells are fixed, stained, and enumerated microscopically or are enumerated via flow cytometry with counting beads. Negative controls are performed with deprivation medium without chemokine, and positive controls are performed with medium for cell culturing containing 10% FBS.

For the in vivo assay, immunodeficient mice engrafted with human melanoma cell line that expresses HMW-MAA are used as subjects. Cells are administered through adoptive transfer with multiple doses of intravenous administration.

To examine the location of adoptively transferred CAR T cells in established melanoma, a readout assay is done via immunohistochemistry. Donor T cells are identified via anti-CD3 and CAR T cells are identified with anti-mouse IgG. qRT-PCR or PCR for detection of migrated CAR T cells in the tissue is also performed.

To examine the antigen-specific immune response establishment in the melanoma, a readout assay is done via flow cytometry of CAR T cells isolated from the melanoma. Phenotypic characterization of surface markers CD3, anti-mouse IgG, CCR10 (or CCR4), CLA, CD69, and HLA-DR is performed. Functional evaluation is done to assay proliferation, Ki67 expression, and numerization of CAR T cells. Cytokine production is evaluated by intracellular cytokine staining upon stimulation.

To examine tumor killing activity of CAR T cells in a tumor site, tumor volume and survival are assayed.

### 5.6. Example 6: Generation of Human Skin Homing CAR T Cells

T lymphocytes are obtained from human PBMCs. CAR T cells generation is performed using a CAR composed of humanized mouse anti-human HMW-MAA-scFv and human co-stimulation intracellular domain and CD3ζ. The gastrointestinal homing receptors are introduced via lentiviral transduction of human CCR10 or CCR4, and CLA nucleic acid sequences. Treatment with 1,25(OH)₂VD₃ and IL-12 is performed during T cell activation. Validation of the CAR T cells is done via phenotypic characterization and functional evaluation via a human CCL27 chemotaxis assay, as in Example 5.

### 5.7. Example 7: Treatment of Gastrointestinal Tumor

An individual presents with a gastrointestinal tumor, for example, adenomatous polyposis coli. Testing of tumor cells from the individual determines that the tumor cells express CEA. T lymphocytes are obtained from the individual, transfected with a lentiviral vector comprising a nucleotide sequence that encodes a chimeric antigen receptor (CAR), and transfected with a second lentiviral vector comprising a nucleotide sequence encoding human CCR9 and/or α4β7 nucleic acid sequences. The T lymphocytes are expanded using CD3+CD28-coated beads in the presence of retinoic acid to sufficient numbers for administration. The chimeric receptor comprises an extracellular antigen-binding region that binds to CEA; a transmembrane domain; an intracellular co-stimulatory domain from CD28; and an intracellular CD3ζ domain. The individual is administered between 10⁹ and 10¹⁰ of the T lymphocytes in 200 mL saline solution by intravenous infusion over 30 minutes. The individual is re-assessed for the gastrointestinal tumor stage and spread to lymph nodes, and histology of biopsied gastrointestinal tissue is performed at 30, 60, and 90 days post-administration.

### 5.8. Example 8: Treatment of Melanoma

An individual presents with melanoma. Testing of tumor cells from the individual determines that the tumor cells express HMW-MAA. T lymphocytes are obtained from the individual, transfected with a lentiviral vector comprising a nucleotide sequence that encodes a chimeric antigen receptor (CAR), and transfected with a second lentiviral vector comprising a nucleotide sequence encoding human CCR10 (or CCR4) and CLA nucleic acid sequences. The T lymphocytes are expanded using CD3+CD28-coated beads in the presence of 1,25(OH)₂VD₃ and IL-12 to sufficient numbers for administration. The chimeric receptor comprises an extracellular antigen-binding region that binds to HMW-MAA; a transmembrane domain; an intracellular co-stimulatory domain from CD28; and an intracellular CD3ζ domain. The individual is administered between 10⁹ and 10¹⁰ of the T lymphocytes in 200 mL saline solution by intravenous infusion over 30 minutes. The individual is re-assessed for the melanoma stage and spread to lymph nodes, and histology of biopsied skin tissue is performed at 30, 60, and 90 days post-administration.

### EQUIVALENTS

The present disclosure is not to be limited in scope by the specific embodiments described herein. Indeed, various modifications of the subject matter provided herein, in addition to those described, will become apparent to those skilled in the art from the foregoing description. Such modifications are intended to fall within the scope of the appended claims.

Various publications, patents and patent applications are cited herein, the disclosures of which are incorporated by reference in their entireties.

The T lymphocytes of the invention are for use in the methods disclosed herein

1. A T lymphocyte expressing a homing receptor not normally expressed by the T lymphocyte, and a chimeric antigen receptor (CAR).

2. The T lymphocyte of claim 1, wherein the homing receptor is:
a B cell zone homing receptor;
a gastrointestinal homing receptor; or
a skin homing receptor.

3. The T lymphocyte of claim 2, wherein:
the B cell zone homing receptor is CXCR5;
the gastrointestinal homing receptor is α4β7 or CCR9; and
the skin homing receptor is CLA, CCR4, or CCR10.

4-6. (canceled)

7. The T lymphocyte of claim 2, further comprising a second gastrointestinal homing receptor;
a second skin homing receptor; or
a second skin homing receptor and a third skin homing receptor.

8. The T lymphocyte of claim 1, wherein the T lymphocyte is activated, expanded, or both activated and expanded in the presence of:
a Vitamin A metabolite, for a time and in an amount sufficient to cause increased expression of one or more gastrointestinal homing receptors, wherein the Vitamin A metabolite is retinoic acid; or
a Vitamin D metabolite and/or IL-12 for a time and in an amount sufficient to cause increased expression of one or more skin homing receptors, the Vitamin D metabolite is 1,26-dihydroxycholecalciferol (1,25(OH)₂D₃).

9-20. (canceled)

21. A method of generating a population of T lymphocytes, wherein a plurality of the T lymphocytes home to the B cell zone of the lymph nodes, the method comprising engineering a population of T lymphocytes to express a B cell zone homing receptor, wherein the B cell homing receptor is CXCR5.

22. (canceled)

23. A method of generating a population of T lymphocytes, wherein a plurality of the T lymphocytes home to the gastrointestinal tract, the method comprising engineering a population of T lymphocytes to express a gastrointestinal homing receptor, wherein the gastrointestinal homing receptor is α4β7 or CCR9.

24-26. (canceled)

27. The method of claim 23, the method further comprising a step wherein the population of T lymphocytes is activated, expanded, or both activated and expanded in the presence of a Vitamin A metabolite for a time and in an amount sufficient to cause increased expression of one or more gastrointestinal homing receptors, wherein the Vitamin A metabolite is retinoic acid.

28. (canceled)

29. A method of generating a population of T lymphocytes, wherein a plurality of the T lymphocytes home to skin tissue or cells, the method comprising engineering a population of T lymphocytes to express a skin homing receptor, wherein the skin homing receptor is CLA, CCR4, or CCR10.

30-34. (canceled)

35. The method of claim 29, wherein the method further comprises a step wherein the population of T lymphocytes is activated, expanded, or both activated and expanded in the presence of a Vitamin D metabolite and/or IL-12 for a time and in an amount sufficient to cause increased expression of one or more skin homing receptors, wherein the Vitamin D metabolite is 1,26-dihydroxycholecalciferol (1,25(OH)₂D₃).

36-37. (canceled)

38. The method of claim 23, wherein the method further comprises a step of engineering the population of T lymphocytes to express a B cell zone homing receptor, wherein the B cell zone homing receptor is CXCR5.

39. (canceled)

40. The method of claim 21, wherein the method further comprises a step of administering a lentiviral vector encoding a chimeric antigen receptor (CAR).

41. A method of treating a cancer or tumor in an individual comprising administering to an individual in need thereof a T lymphocyte comprising (i) a B cell zone homing receptor, wherein the B cell zone homing receptor is CXCR5; and (ii) a CAR.

42. (canceled)

43. A method of treating a gastrointestinal cancer or tumor in an individual comprising administering to an individual in need thereof a T lymphocyte comprising (i) a gastrointestinal homing receptor, wherein the gastrointestinal homing receptor is α4β7 or CCR9; and (ii) a CAR, wherein the extracellular domain of the CAR binds an antigen associated with a gastrointestinal tumor or cancer.

44-47. (canceled)

48. A method of treating a skin cancer or a skin tumor in an individual comprising administering to an individual in need thereof a T lymphocyte comprising (i) a skin homing receptor, wherein the skin homing receptor is CLA, CCR4, or CCR10; and (ii) a CAR, wherein the extracellular domain of the CAR binds an antigen associated with a skin tumor or cancer.

49-55. (canceled)

56. The method of claim 43, wherein the T lymphocyte further comprises a B cell zone homing receptor, wherein the B cell zone homing receptor is CXCR5.

57-74. (canceled)

75. The method of claim 23, wherein the method further comprises a step of administering a lentiviral vector encoding a chimeric antigen receptor (CAR).

76. The method of claim 29, wherein the method further comprises a step of administering a lentiviral vector encoding a chimeric antigen receptor (CAR).

77. The method of claim 29, wherein the method further comprises a step of engineering the population of T lymphocytes to express a B cell zone homing receptor, wherein the B cell zone homing receptor is CXCR5.

78. The method of claim 48, wherein the T lymphocyte further comprises a B cell zone homing receptor, wherein the B cell zone homing receptor is CXCR5.
